(19) United States
(12) Patent Application Publication
(10) Pub. No.: US 2006/0058315 A1
(43) Pub. Date: Mar. 16, 2006

(54) 2-Oxo-ethanesulfonamide derivates

(75) Inventors: Peter John Barton, Macclesfield (GB); David Stephen Clarke, Macclesfield (GB); Craig Samuel Donald, Macclesfield (GB); Janet Elizabeth Pease, Macclesfield (GB)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/533,341

(22) PCT Filed: Nov. 4, 2003
(86) PCT No.: PCT/GB03/04766

§ 371 Date: Apr. 29, 2005

(30) Foreign Application Priority Data
Nov. 7, 2002 (GB) 0225987.7
May 13, 2003 (GB) 0310932.9

(51) Int. Cl. A61K 31/4965; A61K 31/455; A61K 31/426; A61K 31/18

(52) U.S. Cl. 514/255.05; 514/355; 514/365; 514/602; 544/406; 546/315; 564/86; 548/200

(57) ABSTRACT

Compounds of formula (I): Formula (I) wherein variable groups are as defined within; for use in the inhibition of 11βHSD1 are described.

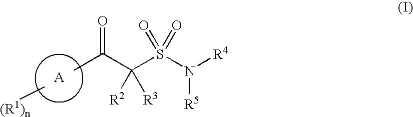

This invention relates to chemical compounds, or pharmaceutically acceptable salts thereof. These compounds possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

Glucocorticoids (cortisol in man, corticosterone in rodents) are counter regulatory hormones i.e. they oppose the actions of insulin (Dallman M F, Strack A M, Akana S F et al. 1993; Front Neuroendocrinol 14, 303-347). They regulate the expression of hepatic enzymes involved in gluconeogenesis and increase substrate supply by releasing glycerol from adipose tissue (increased lipolysis) and amino acids from muscle (decreased protein synthesis and increased protein degradation). Glucocorticoids are also important in the differentiation of pre-adipocytes into mature adipocytes which are able to store triglycerides (Bujalska I J et al. 1999; Endocrinology 140, 3188-3196). This may be critical in disease states where glucocorticoids induced by “stress” are associated with central obesity which itself is a strong risk factor for type 2 diabetes, hypertension and cardiovascular disease (Bjomtorp P & Rosmond R 2000; Int. J. Obesity 24, S80-S85)

It is now well established that glucocorticoid activity is controlled not simply by secretion of cortisol but also at the tissue level by intracellular interconversion of active cortisol and inactive cortisone by the 11-beta hydroxysteroid dehydrogenases, 11βHSD1 (which activates cortisone) and 11βHSD2 (which inactivates cortisol) (Sandeep T C & Walker B R 2001 Trends in Endocrinol & Metab. 12, 446-453). That this mechanism may be important in man was initially shown using carbenoxolone (an anti-ulcer drug which inhibits both 11βHSD1 and 2) treatment which (Walker B R et al. 1995; J. Clin. Endocrinol. Metab. 80, 3155-3159) leads to increased insulin sensitivity indicating that 11βHSD1 may well be regulating the effects of insulin by decreasing tissue levels of active glucocorticoids (Walker B R et al. 1995; J. Clin. Endocrinol. Metab. 80, 3155-3159).

Clinically, Cushing's syndrome is associated with cortisol excess which in turn is associated with glucose intolerance, central obesity (caused by stimulation of pre-adipocyte differentiation in this depot), dyslipidaenia and hypertension. Cushing's syndrome shows a number of clear parallels with metabolic syndrome. Even though the metabolic syndrome is not generally associated with excess circulating cortisol levels (Jessop D S et al. 2001; J. Clin. Endocrinol. Metab. 86, 4109-4114) abnormally high 11βHSD1 activity within tissues would be expected to have the same effect. In obese men it was shown that despite having similar or lower plasma cortisol levels than lean controls, 11βHSD1 activity in subcutaneous fat was greatly enhanced (Rask E et al. 2001; J. Clin. Endoclinol. Metab. 1418-1421). Furthermore, the central fat, associated with the metabolic syndrome expresses much higher levels of 11βHSD1 activity than subcutaneous fat (Bujalska I J et al. 1997; Lancet 349, 1210-1213). Thus there appears to be a link between glucocorticoids, 11βHSD1 and the metabolic syndrome.

11βHSD1 knock-out mice show attenuated glucocorticoid-induced activation of gluconeogenic enzymes in response to fasting and lower plasma glucose levels in response to stress or obesity (Kotelevtsev Y et al. 1997; Proc. Natl. Acad. Sci USA 94, 14924-14929) indicating the utility of inhibition of 11βHSD1 in lowering of plasma glucose and hepatic glucose output in type 2 diabetes. Furthermore, these mice express an anti-atherogenic lipoprotein profile, having low triglycerides, increased HDL cholesterol and increased apo-lipoprotein AI levels. (Morton N M et al. 2001; J. Biol. Chem. 276, 41293-41300). This phenotype is due to an increased hepatic expression of enzymes of fat catabolism and PPARα. Again this indicates the utility of 11βHSD1 inhibition in treatment of the dyslipidaemia of the metabolic syndrome.

The most convincing demonstration of a link between the metabolic syndrome and 11βHSD1 comes from recent studies of transgenic mice over-expressing 11βHSD1 (Masuzaki H et al. 2001; Science 294, 2166-2170). When expressed under the control of an adipose specific promoter, 11βHSD1 transgenic mice have high adipose levels of corticosterone, central obesity, insulin resistant diabetes, hyperlipidaemia and hyperphagia. Most importantly, the increased levels of 11βHSD1 activity in the fat of these mice are similar to those seen in obese subjects. Hepatic 11βHSD1 activity and plasma corticosterone levels were normal, however, hepatic portal vein levels of corticosterone were increased 3 fold and it is thought that this is the cause of the metabolic effects in liver.

Overall it is now clear that the complete metabolic syndrome can be mimicked in mice simply by overexpressing 11βHSD1 in fat alone at levels similar to those in obese man.

11βHSD1 tissue distribution is widespread and overlapping with that of the glucocorticoid receptor. Thus, 11βHSD1 inhibition could potentially oppose the effects of glucocorticoids in a number of physiological/pathological roles. 11βHSD1 is present in human skeletal muscle and glucocorticoid opposition to the anabolic effects of insulin on protein turnover and glucose metabolism are well documented (Whorwood C B et al. 2001; J. Clin. Endocrinol. Metab. 86, 2296-2308). Skeletal muscle must therefore be an important target for 11βHSD1 based therapy.

Glucocorticoids also decrease insulin secretion and this could exacerbate the effects of glucocorticoid induced insulin resistance. Pancreatic islets express 11βHSD1 and carbenoxolone can inhibit the effects of 11-dehydocorticosterone on insulin release (Davani B et al. 2000; J. Biol. Chem. 275, 34841-34844). Thus in treatment of diabetes 11βHSD1 inhibitors may not only act at the tissue level on insulin resistance but also increase insulin secretion itself.

Skeletal development and bone function is also regulated by glucocorticoid action. 11βHSD1 is present in human bone osteoclasts and osteoblasts and treatment of healthy volunteers with carbenoxolone showed a decrease in bone resorption markers with no change in bone formation markers (Cooper M S et al 2000; Bone 27, 375-381). Inhibition of 11βHSD1 activity in bone could be used as a protective mechanism in treatment of osteoporosis.

Glucocorticoids may also be involved in diseases of the eye such as glaucoma. 11βHSD1 has been shown to affect intraocular pressure in man and inhibition of 11βHSD1 may be expected to alleviate the increased intraocular pressure associated with glaucoma (Rauz S et al. 2001; Investigative Opthalmology & Visual Science 42, 2037-2042).

There appears to be a convincing link between 11βHSD1 and the metabolic syndrome both in rodents and in humans. Evidence suggests that a drug which specifically inhibits 11βHSD1 in type 2 obese diabetic patients will lower blood glucose by reducing hepatic gluconeogenesis, reduce central obesity, improve the atherogenic lipoprotein phenotype, lower blood pressure and reduce insulin resistance. Insulin effects in muscle will be enhanced and insulin secretion from the beta cells of the islet may also be increased.

Currently there are two main recognised definitions of metabolic syndrome.

1) The Adult Treatment Panel (ATP III 2001 JMA) definition of metabolic syndrome indicates that it is present if the patient has three or more of the following symptoms:

Waist measuring at least 40 inches (102 cm) for men, 35 inches (88 cm) for women;
Serum triglyceride levels of at least 150 mg/dl (1.69 mmol/l);
HDL cholesterol levels of less than 40 mg/dl (1.04 mmol/l) in men, less than 50 mg/dl (1.29 mmol/l) in women;
Blood pressure of at least 135/80 mm Hg; and/or
Blood sugar (serum glucose) of at least 110 mg/dl (6.1 mmol/l).
   2) The WHO consultation has recommended the following definition which does not imply causal relationships and is suggested as a working definition to be improved upon in due course:
The patient has at least one of the following conditions: glucose intolerance, impaired glucose tolerance (IGT) or diabetes mellitus and/or insulin resistance; together with two or more of the following:
Raised Arterial Pressure;
Raised plasma triglycerides
Central Obesity
Microalbuminuria

We have found that the compounds defined in the present invention, or a pharmaceutically acceptable salt thereof, are effective 11βHSD1 inhibitors, and accordingly have value in the treatment of disease states associated with metabolic syndrome.

Accordingly there is provided the use of a compound of formula (1): 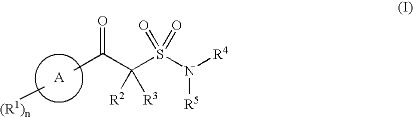
wherein:

Ring A is selected from carbocyclyl or heterocyclyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₙ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino, tri-(C₁₋₄alkyl)silyloxy, carbocyclyl, heterocyclyl, carbocyclylC₀₋₄alkylene-Y— and heterocyclylC₀₋₄alkylene-Y—; wherein R¹ may be optionally substituted on carbon by one or more groups selected from R⁶; and wherein if said heterocyclyl contains an —NH— moiety that nitrogen may be optionally substituted by a group selected from R⁷;

n is 0-5; wherein the values of R¹ may be the same or different;

R² and R³ ale independently selected from hydrogen, hydroxy, amino, cyano, C₁₋₄alkyl, C₁₋₄alkoxy, N-(C₁₋₄alkyl)amino, N,N-(C₁₋₄alkyl)₂amino, carbocyclyl, heterocyclyl, carbocyclylC₁₋₄alkyl, heterocyclylC₁₋₄alkyl; or R² and R³ together form C₂₋₆alkylene; wherein R² and R³ may be independently optionally substituted on carbon by one or more groups selected from R⁸; and wherein if said heterocyclyl contains an —NH— moiety that nitrogen may be optionally substituted by a group selected from R⁹;

one of R⁴ and R⁵ is selected from C₁₋₄alkyl and the other is selected from hydrogen or C₁₋₄alkyl; wherein R⁴ and R⁵ may be optionally substituted on carbon by one or more groups selected from R¹⁰;

Y is —S(O)ₐ—, —O—, —NR¹²—, —C(O), —C(O)NR¹³—, —NR¹⁴C(O)— or —SO₂NR¹⁵—; wherein a is 0 to 2;

R¹², R¹³, R¹⁴ and R¹⁵ are independently selected from hydrogen, phenyl and C₁₋₄alkyl;

R⁶ and R⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino, carbocyclyl and heterocyclyl; wherein R⁶ and R⁸ may be independently optionally substituted on carbon by one or more R¹¹;

R¹⁰ is selected from halo, nitro, cyano, hydroxy, amino, carboxy, carbamoyl, mercapto, sulphamoyl, trifluoromethyl, trifluoromethoxy, C₁₋₄-alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino; wherein R¹⁰ may be independently optionally substituted on carbon by one or more R¹⁶;

R⁷ and R⁹ are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)₂carbamoyl, benzyl, benzyloxycarbonyl, benzoyl and phenylsulphonyl;

R¹¹ and R¹⁶ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

in the manufacture of a medicament for use in the inhibition of 11βHSD1.

According to a further feature of the invention there is provided the use of a compound of formula (I): 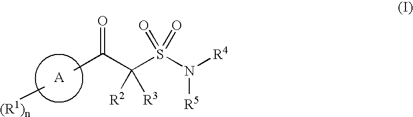
wherein:

Ring A is selected from aryl or heteroaryl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulplhonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R⁶; and wherein if said heterocyclyl contains an —NH— moiety that nitrogen may be optionally substituted by a group selected from R⁷;

n is 0-3; wherein the values of R¹ may be the same or different;

R² and R³ are independently selected from hydrogen, hydroxy, amino, cyano, C₁₋₄alkyl, C₁₋₄alkoxy, N-(C₁₋₄alkyl)amino, N,N-(C₁₋₄alkyl)₂amino, carbocyclyl, heterocyclyl, carbocyclylC₁₋₄alkyl, heterocyclylC₁₋₄alkyl; wherein R² and R³ may be independently optionally substituted on carbon by one or more groups selected from R⁸; and wherein if said heterocyclyl contains an —NH— moiety that nitrogen may be optionally substituted by a group selected from R⁹;

R⁴ and R⁵ are independently selected from C₁₋₄alkyl; wherein R⁴ and R⁵ may be optionally substituted on carbon by one or more groups selected from R¹⁰;

Y is —S(O)ₐ, —O—, —NR¹², —C(O), —C(O)NR¹³—, —NR¹⁴C(O)— or —SO₂NR¹⁵—; wherein a is to 2;

R¹², R¹³, R¹⁴ and R¹⁵ are independently selected from hydrogen, phenyl and C₁₋₄alkyl;

R⁶ and R⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄-alkyl)sulphamoyl, N,N-(C₁₋₄alkyl)₂sulphamoyl, C₁₋₄alkylsulphonylamino, carbocyclyl and heterocyclyl; wherein R⁶ and R⁸ may be independently optionally substituted on carbon by one or more R¹¹;

R¹⁰ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino; wherein R¹⁰ may be independently optionally substituted on carbon by one or more R¹⁶;

R⁷ and R⁹ are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)₂carbamoyl, benzyl, benzyloxycarbonyl, benzoyl and phenylsulphonyl;

R¹¹ and R¹⁶ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

in the manufacture of a medicament for use in the inhibition of 11βHSD1.

According to a further feature of the invention there is provided the use of a compound of formula (I): 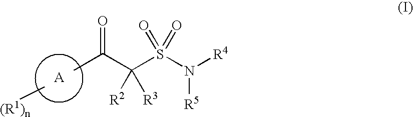
wherein:

Ring A is selected from carbocyclyl or heterocyclyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkyl S(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino, tri-(C₁₋₄alkyl)silyloxy, carbocyclyl, heterocyclyl, carbocyclylC₀₋₄alkylene-Y— and heterocyclylC₀₋₄alkylene-Y—; wherein R¹ may be optionally substituted on carbon by one or more groups selected from R⁶; and wherein if said heterocyclyl contains an —NH— moiety that nitrogen may be optionally substituted by a group selected from R⁷;

n is 0-3; wherein the values of R¹ may be the same or different;
R² and R³ are independently selected from hydrogen, hydroxy, amino, cyano, C₄alkyl, C₁₋₄alkoxy, N-(C₁₋₄alkyl)amino, N,N-(C₁₋₄alkyl)₂amino, carbocyclyl, heterocyclyl, carbocyclylC₁₋₄alkyl, heterocyclylC₁₋₄alkyl; wherein R² and R³ may be independently optionally substituted on carbon by one or more groups selected from R⁸; and wherein if said heterocyclyl contains an —NH— moiety that nitrogen may be optionally substituted by a group selected from R⁹;

one of R⁴ and R⁵ is selected from C₁₋₄alkyl and the other is selected from hydrogen or C₁₋₄alkyl; wherein R⁴ and R⁵ may be optionally substituted on carbon by one or more groups selected from R¹⁰;

Y is —S(O)ₐ—, —O—, —NR¹²—, —C(O), —C(O)NR¹³—, —NR¹⁴C(O)— or —SO₂NR¹⁵—; wherein a is 0 to 2;
R¹², R¹³, R¹⁴ and R¹⁵ are independently selected from hydrogen, phenyl and C₁₋₄alkyl;
R⁶ and R⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino, carbocyclyl and heterocyclyl; wherein R⁶ and R⁸ may be independently optionally substituted on carbon by one or more R¹¹;
R¹⁰ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino; wherein R¹⁰ may be independently optionally substituted on carbon by one or more R¹⁶;

R⁷ and R⁹ are independently selected from C₁₋₄alkyl, C₁₋₄alkanoyl, C₁₋₄-alkylsulphonyl, C₁₋₄alkoxycarbonyl, carbamoyl, N-(C₁₋₄alkyl)carbamoyl, N,N-(C₁₋₄alkyl)₂carbamoyl, benzyl, benzyloxycarbonyl, benzoyl and phenylsulphonyl;

R¹¹ and R¹⁶ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

in the manufacture of a medicament for use in the inhibition of 11βHSD1.

According to a further feature of the invention there is provided a compound of formula (Ia): 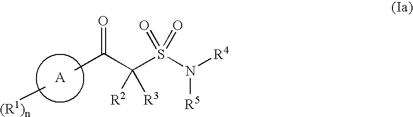
wherein:

Ring A is selected from phenyl, pyridyl, thiazolyl, thienyl and furyl;

R¹ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino; wherein R¹ may be optionally substituted on carbon by one or more groups selected from R⁶; and wherein if said heterocyclyl contains an —NH— moiety that nitrogen may be optionally substituted by a group selected from R⁷;

n is 0-3; wherein the values of R¹ may be the same or different;

R² and R³ are independently selected from hydrogen, hydroxy, amino, cyano, C₁₋₄alkyl, C₁₋₄alkoxy, N-(C₁₋₄alkyl)amino, N,N-(C₁₋₄alkyl)₂amino, carbocyclyl, heterocyclyl, carbocyclylC₁₋₄alkyl, heterocyclylC₁₋₄alkyl; wherein R² and R³ may be independently optionally substituted on carbon by one or more groups selected from R⁸; and wherein if said heterocyclyl contains an —NH— moiety that nitrogen may be optionally substituted by a group selected from R⁹;

R⁴ and R⁵ are independently selected from C₁₋₄alkyl; wherein R⁴ and R⁵ may be optionally substituted on carbon by one or more groups selected from R¹⁰;

R⁶ and R⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino; wherein R⁶ and R⁸ may be independently optionally substituted on carbon by one or more R¹¹;

R¹⁰ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino; wherein R¹⁰ may be independently optionally substituted on carbon by one or more R¹⁶;

R⁷ and R⁹ are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)₂carbamoyl, benzyl, benzyloxycarbonyl, benzoyl and phenylsulphonyl;

R¹¹ and R¹⁶ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof; with the proviso that said compound is not (N-methyl-N-butylsulphamoylmethyl)(phenyl)ketone; [1-(N,N-dimethylsulphamoyl)ethyl](phenyl)ketone; (N,N-dimethylsulphamoylmethyl)(4-nitrophenyl)ketone; (N,N-dimethylsulphamoylmethyl)(4-fluoro-2-methylaminophenyl)ketone; (N,N-dimethylsulphamoylmethyl)(3-methoxy-4-methyl-6-aminophenyl)ketone; (N,N-dimethylsulphamoylmethyl)(3-methoxy-6-aminophenyl)ketone; (N,N-dimethylsulphamoylmethyl)(phenyl)ketone; (N,N-dimethylsulphamoylmethyl)(2-nitro-4-methoxyphenyl)ketone; (N,N-dimethylsulphamoylmethyl)(2-aminomethoxyphenyl)ketone; [1-(N-methyl-N-butylsulphamoyl)ethyl](phenyl)ketone; or (N,N-dimethylsulphamoylmethyl)(thien-2-yl)ketone.

In this specification the term “alkyl” includes both straight and branched chain alkyl groups but references to individual alkyl groups such as “propyl” are specific for the straight chain version only. For example, “C₁₋₄alkyl” includes propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals therefore “carbocyclylC₁₋₄alkyl” includes 1-carbocyclylpropyl, 2-carbocyclylethyl and 3-carbocyclylbutyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

“Heteroaryl” is a totally unsaturated, mono or bicyclic ring containing 3-12 atoms of which at least one atom is chosen from nitrogen, sulphur or oxygen, which may, unless otherwise specified, be carbon or nitrogen linked. Suitably “heteroaryl” refers to a totally unsaturated, monocyclic ring containing 5 or 6 atoms or a bicyclic ring containing 8-10 atoms of which at least one atom is chosen from nitrogen, sulphur or oxygen, which may, unless otherwise specified, be carbon or nitrogen linked. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyranyl, indolyl, pyrimidyl, pyrazinyl, pyridazinyl, benzothienyl, pyridyl and quinolyl. Particularly “heteroaryl” refers to thienyl, furyl, thiazolyl, pyridyl, benzothienyl, imidazolyl or pyrazolyl.

“Aryl” is a totally unsaturated, mono or bicyclic carbon ring that contains 3-12 atoms. Suitably “aryl” is a monocyclic ring containing 5 or 6 atoms or a bicyclic ring containing 9 or 10 atoms. Suitable values for “aryl” include phenyl or naphthyl. Particularly “aryl” is phenyl.

A “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂—group can optionally be replaced by a —C(O)— or a ring sulphur atom may be optionally oxidised to form the S-oxides. Preferably a “heterocyclyl” is a saturated, partially saturated or unsaturated, mono or bicyclic ring containing 5 or 6 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form S-oxide(s). Examples and suitable values of the term “heterocyclyl” are thienyl, piperidinyl, morpholinyl, furyl, thiazolyl, pyridyl, imidazolyl, 1,2,4-triazolyl, thiomorpholinyl, coumarinyl, pyrimidinyl, phthalidyl, pyrazolyl, pyrazinyl, pyridazinyl, benzothienyl, benzimidazolyl, tetrahydrofuryl, [1,2,4]triazolo[4,3-a]pyrimidinyl, piperidinyl, indolyl, 1,3-benzodioxolyl and pyrrolidinyl.

A “carbocyclyl” is a saturated, partially saturated or unsaturated, mono or bicyclic carbon ring that contains 3-12 atoms; wherein a —CH₂— group can optionally be replaced by a —C(O)—. Preferably “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. Particularly “carbocyclyl” is cyclohexyl, phenyl, naphthyl or 2-6-dioxocyclohexyl.

An example of “C₁₋₄alkanoyloxy” is acetoxy. Examples of “C₁₋₄alkoxycarbonyl” include methoxycarbonyl, ethoxycarbonyl, n- and t-butoxycarbonyl. Examples of “C₁₋₄alkoxy” include methoxy, ethoxy and propoxy. Examples of “oxyC₁₋₄alkoxy” include oxymethoxy, oxyethoxy and oxypropoxy. Examples of “C₁₋₄alkanoylamino” include formamido, acetamido and propionylamino. Examples of and “C₁₋₄alkylS(O)ₐ wherein a is 0 to 2” include methylthio, ethylthio, methylsulphinyl, ethylsulphinyl, mesyl and ethylsulphonyl. Examples of and “C₁₋₄alkylsulphonyl” include mesyl and ethylsulphonyl. Examples of “C₁₋₄alkanoyl” include propionyl and acetyl. Examples of “N-(C₁₋₄alkyl)amino” include methylamino and ethylamino. Examples of “N,N-(C₁₋₄alkyl)₂amino” include di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N-(C₁₋₄alkyl)sulphamoyl” are N-(C₁₋₃alkyl)sulphamoyl, N-(methyl)sulphamoyl and N-(ethyl)sulphamoyl. Examples of “N-(C₁₋₄alkyl)₂sulphamoyl” are N,N-(dimethyl)sulphamoyl and N-(methyl)-N-(ethyl)sulphamoyl. Examples of “N-(C₁-alkyl)carbamoyl” are methylaminocarbonyl and ethylaminocarbonyl. Examples of “N,N-(C₁₋₄alkyl)₂carbamoyl” are dimethylaminocarbonyl and methylethylaminocarbonyl. Examples of “C₁₋₄alkylsulphonylamino” are mesylamino and ethylsulphonylamino. Examples of “C₀₋₄alkylene” are a direct bond, methylene and ethylene. Examples of “tri-(C₁₋₄alkyl)silyloxy” include tri-(methyl)silyloxy dimethyl-t-butylsilyloxy. Examples of “C₂₋₅alkylene” are propylene and butylene.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess 11βHSD1 inhibitory activity.

The invention relates to any and all tautomeric forms of the compounds of the formula (I) that possess 11βHSD1 inhibitory activity.

It is also to be understood that certain compounds of the formula (1) can exist in solvated as well as unsolvated forms such as, for example, hydrated forms. It is to be understood that the invention encompasses all such solvated forms which possess 11βHSD1 inhibitory activity.

Particular values of variable groups are as follows. Such values may be used where appropriate with any of the definitions, claims or embodiments defined hereinbefore or hereinafter.

Ring A is selected from aryl.

Ring A is heteroaryl.

Ring A is carbocyclyl.

Ring A is heterocyclyl.

Ring A is phenyl.

Ring A is pyridyl, phenyl, thienyl, furyl or pyrazinyl.

Ring A is pyrid-2-yl, phenyl, thien-2-yl, fur-2-yl, pyrazin-2-yl.

Ring A is pyridyl, phenyl, thienyl, furyl, pyrazinyl, 1,2,3-thiadiazolyl, thiazolyl, cyclohexyl, naphthyl, cyclohexenyl, pyrazolyl, benzothienyl, indolyl, 1,1,3-trioxo-2,3-dihydro-1,2-benzisothiazolyl, 1,3-benzodioxolyl, cyclopentyl, tetrahydropyranyl, 1-oxooctahydropyrido[1,2-a]pyrazinyl, 1,2,3,4-tetrahydronaphthyl, piperidinyl and benzthiazolyl.

Ring A is pyrid-2-yl, pyrid-3-yl, phenyl, thien-2-yl, fur-2-yl, pyrazin-2-yl, 1,2,3-thiadiazol-5-yl, thiazol-2-yl, thiazol-5-yl, cyclohexyl, naphtha-2-yl, cyclohex-1-enyl, pyrazol-3-yl, benzothien-2-yl, indol-5-yl, 1,1,3-trioxo-2,3-dihydro-1,2-benzisothiazol-6-yl, 1,3-benzodioxol-5-yl, cyclopentyl, tetrahydropyran-4-yl, 1-oxooctahydropyrido[1,2-a]pyrazin-7-yl, 1,2,3,4-tetrahydronaphth-2-yl, piperidin-4-yl and benzthiazol-2-yl.

R¹ is selected from halo, cyano, C₁₋₄alkyl, C₂₋₄alkenyl, C₁₋₄alkoxy or C₁₋₄alkanoyl.

R¹ is selected from fluoro, chloro, cyano, methyl, 1-propenyl, methoxy or acetyl.

R¹ is selected from halo, nitro, cyano, sulphamoyl, C₁₋₄alkyl, C₂₋₄alkenyl, C₂₋₄alkynyl, C₁₋₄alkoxy, C₁₋₄alkanoyl, tri-(C₁₋₄alkyl)silyloxy, carbocyclyl and heterocyclylC₀₋₄alkylene-Y—; wherein R¹ may be optionally substituted on carbon by one or more groups selected from R⁶.

R¹ is selected from fluoro, chloro, bromo, iodo, nitro, cyano, sulphamoyl, methyl, ethyl, t-butyl, allyl, ethynyl, methoxy, isopropoxy, acetyl, dimethyl-t-butylsilyloxy, phenyl and pyrimidin-4-ylamino; wherein R¹ may be optionally substituted on carbon by one or more groups selected from R⁶.

R¹ is selected from halo, nitro, cyano, sulphamoyl, C₁₋₄alkyl, C₂₋₄alkenyl, C₂₋₄alkynyl, C₁₋₄alkoxy, C₁₋₄alkanoyl, tri-(C₁₋₄alkyl)silyloxy, carbocyclyl and heterocyclylC₀₋₄alkylene-Y-; wherein R¹ may be optionally substituted on carbon by one or more groups selected from R⁶; wherein

Y is —NR¹²—;

R¹² is hydrogen; and

R⁶ is selected from halo, C₂₋₄alkenyl, C₁₋₄alkanoyl, C₁₋₄alkanoylamino and carbocyclyl.

R¹ is selected from fluoro, chloro, bromo, iodo, nitro, cyano, sulphamoyl, methyl, ethyl, t-butyl, allyl, ethynyl, methoxy, isopropoxy, acetyl, dimethyl-t-butylsilyloxy, phenyl and pyrimidin-4-ylamino; wherein R¹ may be optionally substituted on carbon by one or more groups selected from R⁶; wherein

R⁶ is selected from fluoro, chloro, ethenyl, acetyl, acetylamino and phenyl.

R¹ is selected from fluoro, chloro, bromo, iodo, nitro, cyano, sulphamoyl, methyl, allyl, t-butyl, ethynyl, methoxy, isopropoxy, acetyl, allyloxy, trifluoromethyl, phenyl, benzyloxy, 4-chlorophenyl, 3-oxobutyl, 2-chloropyrimidin-4-yl, acetamidomethyl and dimethyl-t-butylsilyloxy.

Y is —NR¹²—.

R¹² is hydrogen.

R⁶ is selected from halo, C₂₋₄alkenyl, C₁₋₄alkanoyl, C₁₋₄alkanoylamino and carbocyclyl.

R⁶ is selected from fluoro, chloro, ethenyl, acetyl, acetylamino and phenyl.

When Ring A is phenyl, R¹ is selected from 2-fluoro, 3-fluoro, 4-fluoro, 2,4-difluoro, 3-chloro, 3-cyano, 4-cyano, 3-methyl, 3-(1-propenyl), 3-methoxy, 4-methoxy or 4-acetyl.

n is 0-3; wherein the values of R¹ may be the same or different.

n is 0-2; wherein the values of R¹ may be the same or different.

n is 0.

n is 1.

n is 2.

n is 3.

R¹, n and Ring A together form phenyl, 2-fluorophenyl, 3-fluorophenyl, 4-fluorophenyl, 3-chlorophenyl, 4-chlorophenyl, 3-bromophenyl, 4-bromophenyl, 3-iodophenyl, 4-iodophenyl, 3-methylphenyl, 4-t-butylphenyl, 3-methoxyphenyl, 4-methoxyphenyl, 3-isopropoxyphenyl, 4-isopropoxyphenyl, 3-cyanophenyl, 4-cyanophenyl, 4-trifluoromethylphenyl, 4-sulphamoylphenyl, 3-nitrophenyl, 4-nitrophenyl, 3-acetylphenyl, 4-acetylphenyl, 3-allylphenyl, 3-allyloxyphenyl, 4-allyloxyphenyl, 4-ethynylplhenyl, 3-benzyloxyphenyl, 4-benzyloxyphenyl, 4-(3-oxobutyl)phenyl, 4-(dimethyl-t-butylsilyloxy)phenyl, 4-(2-chloropyrimidin-4-ylamino)phenyl, 4-(acetamidomethyl)phenyl, 2,4-difluorophenyl, 3,5-dimethylphenyl, 3,5-dibenzyloxyphenyl, 3-methoxy-4-chlorophenyl, 3-fluoro-4-chlorophenyl, 3-cyano-4-methoxyphenyl, 3-iodo-4-methoxyphenyl, 3-nitro-4-chlorophenyl, 3,4,5-trimethoxyphenyl, biphenyl-3-yl, biphenyl-4-yl, cyclohexyl, 6-cyanonaphth-2-yl, cyclohex-1-en-1-yl, cyclopentyl, 3-phenylcyclopentyl, pyzid-2-yl, 2-methylpyrid-5-yl, thien-2-yl, 5-chlorothien-2-yl, 3-chloro-4-methylthien-2-yl, fur-2-yl, pyrazin-2-yl, 1,2,3-thiadiazol-5-yl, thiazol-2-yl, thiazol-5-yl, 4,5-dichlorothiazol-3-yl, 5-(4-chlorophenyl)pyrazol-3-yl, benzothien-2-yl, indol-5-yl, 1,1,3-trioxo-2,3-dihydro-1,2-benzisothiazol-6-yl, 1,3-benzodioxol-5-yl, tetrahydropyranyl, 1-oxooctahydropyrido[1,2-a]pyrazin-7-yl, 1,2,3,4-tetrahydronaphth-2-yl, 1-(pyrid-4-yl)piperidin-4-yl and benzothiazol-2-yl.

R³, n and Ring A together form phenyl, 2-fluorophenyl, 3-fluorophenyl, 4-fluorophenyl, 3-chlorophenyl, 4-chlorophenyl, 3-bromophenyl, 4-bromophenyl, 3-iodophenyl, 4-iodophenyl, 3-methylphenyl, 4-methylphenyl, 4-t-butylphenyl, 3-methoxyphenyl, 4-methoxyphenyl, 3-isopropoxyphenyl, 4-isopropoxyphenyl, 3-cyanophenyl, 4-cyanophenyl, 4-trifluoromethylphenyl, 4-sulphamoylphenyl, 3-nitrophenyl, 4-nitrophenyl, 3-acetylphenyl, 4-acetylphenyl, 3-allylphenyl, 3-allyloxyphenyl, 4-allyloxyphenyl, 4 ethynylphenyl, 3-benzyloxyphenyl, 4-benzyloxyphenyl, 4-(3-oxobutyl)phenyl, 4-(dimethyl-t-butylsilyloxy)phenyl, 4-(2-chloropyzimidin-4-ylamino)phenyl, 4-(acetamidomethyl)phenyl, 2,4-difluorophenyl, 3,5-dimethylphenyl, 3,5-dibenzyloxyphenyl, 3-methoxy-4-chlorophenyl, 3-fluoro-4-chlorophenyl, 3-cyano-4-methoxyphenyl, 3-iodo-4-methoxyphenyl, 3-nitro-4-chlorophenyl, 3,4,5-trimethoxyphenyl, biphenyl-3-yl, biphenyl-4-yl, cyclohexyl, 6-cyanonaphth-2-yl, cyclohex-1-en-1-yl, cyclopentyl, 3-phenylcyclopentyl, pyrid-2-yl, 2-methylpyrid-5-yl, thien-2-yl, 5-chlorothien-2-yl, 3-chloro-methylthien-2-yl, fur-2-yl, pyrazin-2-yl, 1,2,3-thiadiazol-5-yl, thiazol-2-yl, thiazol-5-yl, 4,5-dichlorothiazol-3-yl, 5-(4-chlorophenyl)pyrazol-3-yl, benzothien-2-yl, indol-5-yl, 1,1,3-trioxo-2,3-dihydro-1,2-benzisothiazol-6-yl, 1,3-benzodioxol-5-yl, tetrahydropyran-4-yl, 1-oxooctahydropyrido[1,2-a]pyrazin-7-yl, 1,2,3,4-tetrahydronaphth-2-yl, 1-(pyrid-4-yl)piperidin-4-yl and benzothiazol-2-yl.

R² and R³ are both hydrogen.

R² and R³ are independently selected from hydrogen or C₁₋₄alkyl.

R² and R³ are independently selected from hydrogen or C₁₋₄alkyl, or R² and R³ together form C₂₋₆alkylene.

R² and R³ are independently selected from hydrogen or methyl.

R² and R³ are independently selected from hydrogen or methyl or R² and R³ together form butylene.

R² and R³ are both hydrogen.

R² and R³ are both methyl.

R² and R³ together form butylene.

One of R² and R³ is hydrogen and the other is methyl.

R⁴ and R⁵ are independently selected from C₁₋₄alkyl; wherein R⁴ and R⁵ may be optionally substituted on carbon by one or more groups selected from R¹⁰; and

R¹⁰ is selected from C₁₋₄alkoxy and N,N-(C₁₋₄alkyl)₂amino.

one of R⁴ and R⁵ is selected from hydrogen and C₁₋₄alkyl and the other is selected from C₁₋₄alkyl; wherein R⁴ and R⁵ may be optionally substituted on carbon by one or more groups selected from R¹⁰; and

R¹⁰ is selected from C₁₋₄alkoxy and N,N-(C₁₋₄alkyl)amino.

one of R⁴ and R⁵ is selected from selected from hydrogen, methyl, isopropyl and ethyl, and the other is selected from methyl, isopropyl, propyl and ethyl; wherein R⁴ and R⁵ may be optionally substituted on carbon by one or more groups selected from R¹⁰.

R⁴ and R⁵ are independently selected from methyl, ethyl, propyl and isopropyl; wherein R⁴ and R⁵ may be optionally substituted on carbon by one or more groups selected from R¹⁰; and

R¹⁰ is selected from methoxy and N,N-dimethylamino.

one of R⁴ and R⁵ is selected from selected from hydrogen, methyl, isopropyl and ethyl, and the other is selected from methyl, isopropyl, propyl and ethyl; wherein R⁴ and R⁵ may be to optionally substituted on carbon by one or more groups selected from R¹⁰; and

R¹⁰ is selected from methoxy, isopropoxy and N,N-dimethylamino.

R⁴ and R⁵ are independently selected from methyl, ethyl, 2-methoxyethyl, 2-(N,N-dimethylamino)propyl and isopropyl.

one of R⁴ and R⁵ is selected from selected from hydrogen, methyl, isopropyl and ethyl; and the other is selected from methyl, isopropyl, propyl, 2-methoxyethyl, 2-dimethylaminoethyl, 2-(isopropoxy)ethyl and ethyl.

R¹⁰ is selected from C₁₋₄alkoxy and N,N-(C₁₋₄alkyl)₂amino.

R¹⁰ is selected from methoxy, isopropoxy and N,N-dimethylamino.

R⁴ and R⁵ together with the nitrogen to which they are attached form isopropylamino, dimethylamino, diethylamino, diisopropylamino, N-(methyl)-N-(propyl)amino, N-(methyl)-N-(isopropyl)amino, N-(methyl)-N-(2-methoxyethyl)amino, N-(isopropyl)-N-(2-methoxyethyl)amino, N-(isopropyl)-N-[2-(isopropoxy)ethyl]amino, N-(methyl)-N-(2-dimethylaminoethyl)amino and N-(ethyl)-N-(isopropyl)amino.

Therefore in a further aspect of the invention there is provided the use of a compound of formula (1) (as depicted above) wherein:

Ring A is pyridyl, phenyl, thienyl, furyl or pyrazinyl;

R¹ is selected from halo, cyano, C₁₋₄alkyl, C₂₋₄alkenyl, C₁₋₄alkoxy or C₁₋₄alkanoyl;

n is 0-2; wherein the values of R¹ may be the same or different;

R² and R³ are independently selected from hydrogen or C₁₋₄alkyl;

R⁴ and R⁵ are independently selected from C₁₋₄alkyl; wherein R⁴ and R⁵ may be optionally substituted on carbon by one or more groups selected from R¹⁰; and

R¹⁰ is selected from C₁₋₄alkoxy and N₃N-(C₁₋₄alkyl)₂amino;

or a pharmaceutically acceptable salt thereof;

in the manufacture of a medicament for use in the inhibition of 11βHSD1.

Therefore in a further aspect of the invention there is provided the use of a compound of formula (I) (as depicted above) wherein:

Ring A is pyrid-2-yl, phenyl, thien-2-yl, fur-2-yl, pyrazin-2-yl;

R¹ is selected from fluoro, chloro, cyano, methyl, 1-propenyl, methoxy or acetyl;

n is 0-2; wherein the values of R¹ may be the same or different;

R² and R³ are independently selected from hydrogen or methyl;

R⁴ and R⁵ are independently selected from methyl, ethyl, 2-methoxyethyl, 2-(N,N-dimethylamino)propyl and isopropyl;

or a pharmaceutically acceptable salt thereof;

in the manufacture of a medicament for use in the inhibition of 11βHSD1.

Therefore in a further aspect of the invention there is provided the use of a compound of formula (1) (as depicted above) wherein:

Ring A is carbocyclyl or heterocyclyl;

R¹ is selected from halo, nitro, cyano, sulphamoyl, C₁₋₄alkyl, C₂₋₄alkenyl, C₂₋₄alkynyl, C₁₋₄alkoxy, C₁₋₄alkanoyl, tri-(C₁₋₄alkyl)silyloxy, carbocyclyl and heterocyclylC₀₋₄alkylene-Y—; wherein R¹ may be optionally substituted on carbon by one or more groups selected from R⁶; wherein:

Y is —NR¹²—;

R¹² is hydrogen; and

R⁶ is selected from halo, C₂₋₄alkenyl, C₁₋₄alkanoyl, C₁₋₄alkanoylamino and carbocyclyl;

n is 0-3; wherein the values of R¹ may be the same or different;

R² and R³ are independently selected from hydrogen or C₁₋₄ alkyl;

one of R⁴ and R⁵ is selected from hydrogen and C₁₋₄alkyl and the other is selected from C₁₋₄ alkyl; wherein R⁴ and R⁵ may be optionally substituted on carbon by one or more groups selected from R¹⁰; and

R¹⁰ is selected from C₁₋₄alkoxy and N,N-(C₁₋₄alkyl)₂amino;

or a pharmaceutically acceptable salt thereof;

in the manufacture of a medicament for use in the inhibition of 11βHSD1.

Therefore in a further aspect of the invention there is provided the use of a compound of formula (I) (as depicted above) wherein:

R¹, n and Ring A together form phenyl, 2-fluorophenyl, 3-fluorophenyl, 4-fluorophenyl, 3-chlorophenyl, 4-chlorophenyl, 3-bromophenyl, 4-bromophenyl, 3-iodophenyl, 4-iodophenyl, 3-methylphenyl, 4-t-butylphenyl, 3-methoxyphenyl, 4-methoxyphenyl, 3-isopropoxyphenyl, 4-isopropoxyphenyl, 3-cyanophenyl, 4-cyanophenyl, 4-trifluoromethylphenyl, 4-sulphamoylphenyl, 3-nitrophenyl, 4-nitrophenyl, 3-acetylphenyl, 4-acetylphenyl, 3-allylphenyl, 3-allyloxyphenyl, 4-allyloxyphenyl, 4-ethynylphenyl, 3-benzyloxyphenyl, 4-benzyloxyphenyl, 4-(3-oxobutyl)phenyl, 4-(dimethyl-t-butylsilyloxy)phenyl, 4-(2-chloropyrinidin-4-ylamino)phenyl, 4-(acetamidomethyl)phenyl, 2,4-difluorophenyl, 3,5-dimethylphenyl, 3,5-dibenzyloxyphenyl, 3-methoxy-4-chlorophenyl, 3-fluoro-4-chlorophenyl, 3-cyano-4-methoxyphenyl, 3-iodo-4-methoxyphenyl, 3-nitro-4-chlorophenyl, 3,4,5-trimethoxyphenyl, biphenyl-3-yl, biphenyl-4-yl, cyclohexyl, 6-cyanonaphth-2-yl, cyclohex-1-en-1-yl, cyclopentyl, 3-phenylcyclopentyl, pyrid-2-yl, 2-methylpyrid-5-yl, thien-2-yl, 5-chlorothien-2-yl, 3-chloro-4-methylthien-2-yl, fur-2-yl, pyrazin-2-yl, 1,2,3-thiadiazol-5-yl, thiazol-2-yl, thiazol-5-yl, 4,5-dichlorothiazol-3-yl, 5-(4-chlorophenyl)pyrazol-3-yl, benzothien-2-yl, indol-5-yl, 1,1,3-trioxo-2,3-dihydro-1,2-benzisothiazol-6-yl, 1,3-benzodioxol-5-yl, tetrahydropyran-4-yl, 1-oxooctahydropyrido[1,2-a]pyrazin-7-yl, 1,2,3,4-tetrahydronaphth-2-yl, 1-(pyrid-4-yl)piperidin-4-yl and benzothiazol-2-yl;

R² and R³ are independently selected from hydrogen or methyl; and

R⁴ and R⁵ together with the nitrogen to which they are attached form isopropylamino, dimethylamino, diethylamino, diisopropylamino, N-(methyl)-N-(propyl)amino, N-(methyl)-N-(isopropyl)amino, N-(methyl)-N-(2-methoxyethyl)amino, N-(isopropyl)-N-(2-methoxyethyl)amino, N-(isopropyl)-N-[2-(isopropoxy)ethyl]amino, N-(methyl)-N-(2-dimethylaminoethyl)amino and N-(ethyl)-N-(isopropyl)amino;

or a pharmaceutically acceptable salt thereof;

in the manufacture of a medicament for use in the inhibition of 11βHSD1.

Therefore in a further aspect of the invention there is provided the use of a compound of formula (I) (as depicted above) wherein:

Ring A is carbocyclyl or heterocyclyl;

R¹ is selected from halo, nitro, cyano, sulphamoyl, C₁₋₄alkyl, C₂₋₄alkenyl, C₂₋₄alkynyl, C₁₋₄alkoxy, C₁₋₄alkanoyl, tri-(C₁₋₄alkyl)silyloxy, carbocyclyl and heterocyclylC₀₋₄alkylene-Y-; wherein R¹ may be optionally substituted on carbon by one or more groups selected from R⁶; wherein:

Y is —NR¹²—;

R¹² is hydrogen; and

R⁶ is selected from halo, C₂₋₄alkenyl, C₁₋₄alkanoyl, C₁₋₄alkanoylamino and carbocyclyl;

n is 0-3; wherein the values of R¹ may be the same or different;

R² and R³ are independently selected from hydrogen or C₁₋₄alkyl, or R² and R³ together form C₂₋₆alkylene;

one of R⁴ and R⁵ is selected from hydrogen and C₁₋₄alkyl and the other is selected from C₁₋₄alkyl; wherein R⁴ and R⁵ may be optionally substituted on carbon by one or more groups selected from R¹⁰; and

R¹⁰ is selected from C₁₋₄alkoxy and N,N-(C₁₋₄alkyl)₂amino;

or a pharmaceutically acceptable salt thereof;

in the manufacture of a medicament for use in the inhibition of 11βHSD1.

Therefore in a further aspect of the invention there is provided the use of a compound of formula (I) (as depicted above) wherein:

R¹, n and Ring A together form phenyl, 2-fluorophenyl, 3-fluorophenyl, 4-fluorophenyl, 3-chlorophenyl, 4-chlorophenyl, 3-bromophenyl, 4-bromophenyl, 3-iodophenyl, 4-iodophenyl, 3-methylphenyl, 4-methylphenyl, 4-t-butylphenyl, 3-methoxyphenyl, 4-methoxyphenyl, 3-isopropoxyphenyl, 4 isopropoxyphenyl, 3-cyanophenyl, 4-cyanophenyl, 4-trifluoromethylphenyl, 4-sulphamoylphenyl, 3-nitrophenyl, 4-nitrophenyl, 3-acetylphenyl, 4-acetylphenyl, 3-allylphenyl, 3-allyloxyphenyl, 4-allyloxyphenyl, 4-ethynylphenyl, 3-benzyloxyphenyl, 4-benzyloxyphenyl, 4-(3-oxobutyl)phenyl, 4-(dimethyl-t-butylsilyloxy)phenyl, 4-(2-chloropyrimidin-4-ylamino)phenyl, 4-(acetamidomethyl)phenyl, 2,4-difluorophenyl, 3,5-dimethylphenyl, 3,5-dibenzyloxyphenyl, 3-methoxy-4-chlorophenyl, 3-fluoro-4-chlorophenyl, 3-cyano-4-methoxyphenyl, 3-iodo-4-methoxyphenyl, 3-nitro-4-chlorophenyl, 3,4,5-trimethoxyphenyl, biphenyl-3-yl, biphenyl-4-yl, cyclohexyl, 6-cyanonaphth-2-yl, cyclohex-1-en-1-yl, cyclopentyl, 3-phenylcyclopentyl, pyrid-2-yl, 2-methylpyrid-5-yl, thien-2-yl, 5-chlorothien-2-yl, 3-chloro-4-methylthien-2-yl, fur-2-yl, pyrazin-2-yl, 1,2,3-thiadiazol-5-yl, thiazol-2-yl, thiazol-5-yl, 4,5-dichlorothiazol-3-yl, 5-(4-chlorophenyl)pyrazol-3-yl, benzothien-2-yl, indol-5-yl, 1,1,3-trioxo-2,3-dihydro-1,2-benzisothiazol-6-yl, 1,3-benzodioxol-5-yl, tetrahydropyran-4-yl, 1-oxooctahydropyrido[1,2-a]pyrazin-7-yl, 1,2,3,4-tetrahydronaphth-2-yl, 1-(pyrid-4-yl)piperidin-4-yl and benzothiazol-2-yl.

R² and R³ are independently selected from hydrogen or methyl or R² and R³ together form butylene;

R⁴ and R³ together with the nitrogen to which they are attached form isopropylamino, dimethylamino, diethylamino, diisopropylamino, N-(methyl)-N-(propyl)amino, N-(methyl)-N-(isopropyl)amino, N-(methyl)-N-[2-methoxyethyl)amino, N-(isopropyl)-N-(2-methoxyethyl)amino, N-(isopropyl)-N-[2-(isopropoxy)ethyl]amino, N-(methyl)-N-(2-dimethylaminoethyl)amino and N-(ethyl)-N-(isopropyl)amino;

or a pharmaceutically acceptable salt thereof;

in the manufacture of a medicament for use in the inhibition of 11βHSD1.

In another aspect of the invention, preferred compounds of the invention are any one of the Examples or a pharmaceutically acceptable salt thereof.

In another aspect of the invention, preferred compounds of the invention are Examples 2, 8, 12, 14, 31, 35, 55, 75 or 82, or a pharmaceutically acceptable salt thereof.

In another aspect of the invention, preferred compounds of the invention are any one of the Reference Examples or a pharmaceutically acceptable salt thereof.

Another aspect of the present invention provides a process for preparing a compound of formula (I) or (Ia) or a pharmaceutically acceptable salt thereof which process (wherein variable groups are, unless otherwise specified, as defined in formula (I) or (Ia)) comprises of:
Process 1): Reacting a Compound of Formula (II): 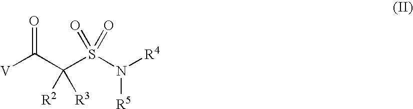
wherein V is a displaceable group; with an organometallic reagent of formula (III): 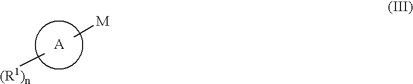
wherein M is a metal reagent;
Process 2): Reacting a Compound of Formula (IV): 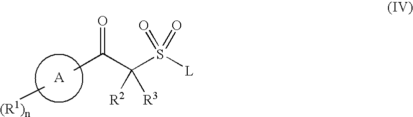
wherein L is a displaceable group; with an amine of formula (V): 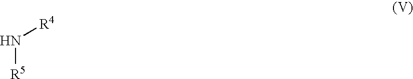
Process 3): Reacting a Compound of Formula (VI): 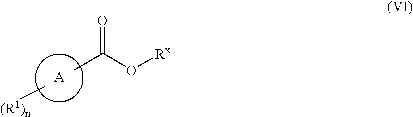
wherein R^{x}OC(O)— is an ester with a compound of formula (VII): 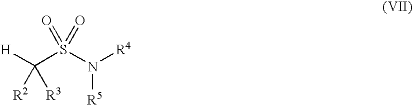
Process 4): for Compounds of Formula (a) wherein R² is not Hydrogen; Deprotonating a Compound of Formula (VIII): 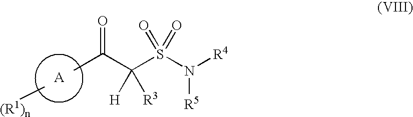
and reacting with a compound of formula (IX):
R²-L (IX)
wherein R² is not hydrogen and L is a displaceable group;
and thereafter if necessary or desirable:
i) converting a compound of the formula (I) into another compound of the formula (I);
i) removing any protecting groups;
ii) forming a pharmaceutically acceptable salt thereof.

L is a displaceable group, suitable values for L include halo, particularly fluoro or chloro.

V is a displaceable group, suitable values for V include the Weinreb amide N-methyl-N-methoxyamine.

M is a metal reagent. Suitable values for M include Grignard reagents such as MgBr and lithium.

The group R^{x}OC(O)— is an ester. Suitable values for R^{x} are methyl and ethyl.

The reactions described above may be performed under standard conditions. The intermediates described above are commercially available, are known in the art or may be prepared by known procedures.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess 11βHSD1 inhibitory activity. These properties may be assessed using the following assay.

Assay

HeLa cells (human cervical carcinoma derived cells) were stably transfected with a construct containing four copies of the glucocorticoid response element (GRE) linked to a beta-galactosidase reporter gene (3 kb lac Z gene derived from pSV-B-galactosidase). These cells were then further stably transfected with a construct containing full-length human 11βHSD1 enzyme (in pCMVHyg) to create GRE4-βGal/11βHSD1 cells. The principal of the assay is as follows. Cortisone is freely taken up by the cells and is converted to cortisol by 11βHSD1 oxo-reductase activity and cortisol (but not cortisone) binds to and activates the glucocorticoid receptor. Activated glucocorticoid receptor then binds to the GRE and initiates transcription and translation of β-galactosidase. Enzyme activity can then be assayed with high sensitivity by colourimetric assay. Inhibitors of 11βHSD1 will reduce the conversion of cortisone to cortisol and hence decrease the production of β-galactosidase.

Cells were routinely cultured in DMIM (Invitrogen, Paisley, Renfrewshire, UK) containing 10% foetal calf serum (LabTech), 1% glutamine (Invitrogen), 1% penicillin & streptomycin (Invitrogen), 0.5 mg/ml G418 (Invitrogen) & 0.5 mg/ml hygromycin (Boehringer). Assay media was phenol red free-DMEM containing 1% glutamine, 1% penicillin & streptomycin.

Compounds (1 mM) to be tested were dissolved in dimethyl sulphoxide (DMSO) and serially diluted into assay media containing 10% DMSO. Diluted compounds were then plated into transparent flat-bottomed 384 well plates (Matrix, Hudson N H, USA).

The assay was carried out in 384 well microtitre plate (Matrix) in a total volume of 50 μl assay media consisting of cortisone (Sigma, Poole, Dorset, UK, 1 μM), HeLa GRE4-βGal/11βHSD1 cells (10,000 cells) plus test compounds (3000 to 0.01 nM). The plates were then incubated in 5% O₂, 95% CO₂ at 37° C. overnight.

The following day plates were assayed by measurement of β-galactosidase production.

A cocktail (25 μl) consisting of 10× Z-buffer (600 mM Na₂HPO₄, 400 mM NaH₂PO₄.2H₂O, 100 mM KCl, 10 mM MgSO₄.7H₂O, 500 mM, β-mercaptoethanol, pH 7.0), SDS (0.2%), chlorophenol red-β-D-galactopyranoside (5 mM, Roche Diagnostics) was added per well and plates incubated at 37° C. for 3-4 hours. β-Galactosidase activity was indicated by a yellow to red colour change (absorbance at 570 nm) measured using a Tecan Spectrafluor Ultra.

The calculation of median inhibitory concentration (IC₅₀) values for the inhibitors was performed using Origin 6.0 (Microcal Software, Northampton M A USA). Dose response curves for each inhibitor were plotted as OD units at each inhibitor concentration with relation to a maximum signal (cortisone, no compound) and IC₅₀ values calculated. Compounds of the present invention typically show an IC₅₀<10 μM. For example the following results were obtained:

| Example | IC₅₀ (nM) |
|---|---|
| 2 | 72 |
| 14 | 47 |
| 75 | 60 |

According to a further aspect of the invention there is provided a pharmaceutical composition which comprises a compound of formula (Ia) or a pharmaceutically acceptable salt thereof, or a compound selected from Examples, or a pharmaceutically acceptable salt thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I), or a pharmaceutically acceptable salt thereof, will normally be administered to a warm-blooded animal at a unit dose within the range 0.1-50 mg/kg that normally provides a therapeutically-effective dose. A unit dose form such as a tablet or capsule will usually contain, for example 1-1000 mg of active ingredient. However the daily dose will necessarily be varied depending upon the host treated, the particular route of administration, and the severity of the illness being treated. Accordingly the optimum dosage may be determined by the practitioner who is treating any particular patient.

We have found that the compounds defined in the present invention, or a pharmaceutically acceptable salt thereof, are effective 11βHSD1 inhibitors, and accordingly have value in the treatment of disease states associated with metabolic syndrome.

It is to be understood that where the term “metabolic syndrome” is used herein, this relates to metabolic syndrome as defined in 1) and/or 2) or any other recognised definition of this syndrome. Synonyms for “metabolic syndrome” used in the art include Reaven's Syndrome, Insulin Resistance Syndrome and Syndrome X. It is to be understood that where the term “metabolic syndrome” is used herein it also refers to Reaven's Syndrome, Insulin Resistance Syndrome and Syndrome X.

According to a further aspect of the present invention there is provided a compound of the formula (Ia) or a pharmaceutically acceptable salt thereof, or a compound selected from Examples, or a pharmaceutically acceptable salt thereof, as defined hereinbefore for use in a method of prophylactic or therapeutic treatment of a warm-blooded animal, such as man.

Thus according to this aspect of the invention there is provided a compound of the formula (Ia) or a pharmaceutically acceptable salt thereof, or a compound selected from Examples, or a pharmaceutically acceptable salt thereof, as defined hereinbefore for use as a medicament.

According to another feature of the invention there is provided the use of a compound of the formula (Ia) or a pharmaceutically acceptable salt thereof, or a compound selected from the Examples, or a pharmaceutically acceptable salt thereof, as defined hereinbefore in the manufacture of a medicament for use in the production of an 11βHSD1 inhibitory effect in a warm-blooded animal, such as man.

According to another feature of the invention there is provided the use of a compound selected from Reference Examples, or a pharmaceutically acceptable salt thereof, as defined hereinbefore in the manufacture of a medicament for use in the production of an 11βHSD1 inhibitory effect in a warm-blooded animal, such as man.

Where production of or producing an 11βHSD1 inhibitory effect is referred to suitably this refers to the treatment of metabolic syndrome. Alternatively, where production of an 11βHSD1 inhibitory effect is referred to this refers to the treatment of diabetes, obesity, hyperlipidaemia, hyperglycaenma, hyperinsulinemia or hypertension, particularly diabetes and obesity. Alternatively, where production of an 11βHSD1 inhibitory effect is referred to this refers to the treatment of glaucoma, osteoporosis, tuberculosis, dementia, cognitive disorders or depression.

According to a further feature of this aspect of the invention there is provided a method for producing an 11βHSD1 inhibitory effect in a warm-blooded animal, such as man, in need of such treatment which comprises administering to said animal an effective amount of a compound of formula (I), or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound of formula (Ia) or a pharmaceutically acceptable salt thereof, or a compound selected from Examples, or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound selected from the Reference Examples, or a pharmaceutically acceptable salt thereof.

In addition to their use in therapeutic medicine, the compounds of formula (I), or a pharmaceutically acceptable salt thereof, are also useful as pharmacological tools in the development and standardisation of in vitro and in vivo test systems for the evaluation of the effects of inhibitors of 11βHSD1 in laboratory animals such as cats, dogs, rabbits, monkeys, rats and mice, as part of the search for new therapeutic agents.

The inhibition of 11βHSD1 described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example agents than might be co-administered with 11βHSD1 inhibitors, particularly those of the present invention, may include the following main categories of treatment:
1) Insulin and insulin analogues;
2) Insulin secretagogues including sulphonylureas (for example glibenclamide, glipizide) and prandial glucose regulators (for example repaglinide, nateglinide);
3) Insulin sensitising agents including PPARγ agonists (for example pioglitazone and rosiglitazone);
4) Agents that suppress hepatic glucose output (for example metformin);
5) Agents designed to reduce the absorption of glucose from the intestine (for example acarbose);
6) Agents designed to treat the complications of prolonged hyperglycaemia; e.g. aldose reductase inhibitors
7) Other anti-diabetic agents including phosotyrosine phosphatase inhibitors, glucose 6-phosphatase inhibitors, glucagon receptor antagonists, glucokinase activators, glycogen phosphorylase inhibitors, fructose 1,6 bisphosphastase inhibitors, glutamine:fructose-6-phosphate amidotransferase inhibitors
8) Anti-obesity agents (for example sibutramine and orlistat);
9) Anti-dyslipidaemia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); ileal bile acid absorption inhibitors (IBATi), cholesterol ester transfer protein inhibitors and nicotinic acid and analogues (niacin and slow release formulations);
10) Antihypertensive agents such as, β blockers (eg atenolol, inderal); ACE inhibitors (eg lisinopril); calcium antagonists (eg. nifedipine); angiotensin receptor antagonists (eg candesartan), α antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin; and
12) Anti-inflammatory agents, such as non-steroidal anti-inflammatory drugs (eg. aspirin) and steroidal anti-inflammatory agents (eg. cortisone).

In the above other pharmaceutical composition, process, method, use and medicament manufacture features, the alternative and preferred embodiments of the compounds of the invention described herein also apply.

### EXAMPLES

The invention will now be illustrated in the following non limiting Examples, in which standard techniques known to the skilled chemist and techniques analogous to those described in these Examples may be used where appropriate, and in which, unless otherwise stated:

(i) evaporations were carried out by rotary evaporation in vacuo and work up procedures were carried out after removal of residual solids such as drying agents by filtration;

(ii) all reactions were carried out under an inert atmosphere at ambient temperature, typically in the range 18-25° C., with solvents of EPLC grade under anhydrous conditions, unless otherwise stated;

(iii) column chromatography (by the flash procedure) was performed on Silica gel 40-63 μm (Merck);

(iv) yields are given for illustration only and are not necessarily the maximum attainable;

(v) the structures of the end products of the formula (I) were generally confirmed by nuclear (generally proton) magnetic resonance (NMR) and mass spectral techniques; magnetic resonance chemical shift values were measured in deuterated CDCl₃ (unless otherwise stated) on the delta scale (ppm downfield from tetramethylsilane); proton data is quoted unless otherwise stated; spectra were recorded on a Varian Mercury-300 MHz, Varian Unity plus-400 MHz, Varian Unity plus-600 MHz or on Varian Inova-500 MHz spectrometer unless otherwise stated data was recorded at 400 Mb; and peak multiplicities are shown as follows: s, singlet; d, doublet; dd, double doublet; t, triplet; tt, triple triplet; q, quartet; tq, triple quartet; m, multiplet; br, broad; ABq, AB quartet; ABd, AB doublet, ABdd, AB doublet of doublets; dABq, doublet of AB quartets; LCMS were recorded on a Waters Z MD, LC column xTerra MS C₈(Waters), detection with a HP 100 MS-detector diode array equipped; mass spectra (MS) (loop) were recorded on VG Platform II (Fisons Instruments) with a HP-1100 MS-detector diode array equipped; unless otherwise stated the mass ion quoted is (MH⁺); unless further details are specified in the text, analytical high performance liquid chromatography (HPLC) was performed on Prep LC 2000 (Waters), Cromasil C₈, 7 μm, (Akzo Nobel); MeCN and de-ionised water 10 mM ammonium acetate as mobile phases, with suitable composition;

(vii) intermediates were not generally fully characterised and purity was assessed by thin layer chromatography (TLC), HPLC, infra-red (IR), MS or NMR analysis;

(viii) where solutions were dried magnesium sulphate was the drying agent;

(ix) where an “ISOLUTE” column is referred to, this means a column containing 2 g of silica, the silica being contained in a 6 ml disposable syringe and supported by a porous disc of 54 Å pore size, obtained from International Sorbent Technology under the name “ISOLUTE”; “ISOLUTE” is a registered trade mark;

(x) the following abbreviations may be used hereinbefore or hereinafter:

DCM dichloromethane;
EtOAc ethyl acetate;
MeCN acetonitrile; and
THF tetrahydrofuran.

### Reference Example 1

### N,N-Dimethylsulphamoylmethyl)(phenyl)ketone

The title compound was prepared by the procedure of J. Med. Chem.; EN; 30; 12; 1987; 2232-2239. Reference Example 1 is exemplified in this reference.

### Reference Example 2

### (N,N-Dimethylsulphamoylmethyl)(4-fluorophenyl)ketone

The title compound was prepared by the procedure of Reference Example 1. NMR: 2.9 (s, 6H), 4.5 (s, 2H), 7.2 (m, 2H), 8.0 (m, 2H); m/z 244.

### Reference Example 3

### (N,N-Dimethylsulphamoylmethyl)(thien-2-yl)ketone

To a stirred solution of methylthiophene-2-carboxylate (520 mg, 3.65 mmol) and N,N-dimethylmethanesulphonamide (375 mg, 3.04 mmol) in ethylene glycol dimethyl ether (15 ml) was added sodium hydride (60% suspension in oil, 328 mg, 8.21 mmol). The reaction was warmed to 85° C. and stirred at this temperature overnight then cooled to room temperature and quenched with water. The resulting brown solution was acidified to ˜pH2 with concentrated hydrochloric acid and then extracted with DCM (2×40 ml). The organic layers were combined, washed with water (30 ml) and brine (20 ml) then dried, filtered and evaporated to yield crude product. This was purified by column chromatography (20 g Silica, eluting with DCM) to yield an oil which crystallised on standing. This material was still impure. The crude was product partitioned between DCM and 1M sodium hydroxide solution, the layers separated and the sodium hydroxide layer re extracted with DCM. The aqueous layer was then acidified to ˜pH3 with concentrated HCl and then extracted with DCM twice. These two DCM layers were combined, washed with brine, dried, filtered and evaporated to yield the product as a solid (56 mg, 7%). NMR: 2.90 (s, 6H), 4.45 (s, 2H), 7.20 (m, 1H), 7.75 (m, 1H), 7.90 (m, 1H); m/z: 234.

### Reference Example 4

### [1-(N,N-Dimethylsulphamoyl)ethyl](phenyl)ketone

To a stirred solution of (N,N-dimethylsulphamoylmethyl)(phenyl)ketone (Reference Example 1; 88 mg, 0.39 mmol) in DMF (7 ml) was added potassium carbonate (107 mg, 0.78 mmol) followed by methyl iodide (113 mg, 0.8 mmol). The resulting suspension was stirred at room temperature for 2 hours. The reaction was quenched with water (˜50 ml) and extracted with DCM (2×50 ml). The organic layers combined, dried, filtered and evaporated to yield the product as a yellow oil (still contains a trace of DMF) NMR: 1.70 (d, 3H), 2.90 (s, 6H), 5.15 (m, 1H), 7.50 (t, 2H), 7.60 (m, 2H), 8.00 (br m, 1H); m/z: 242.

### Example 1

### [1-(N,N-Dimethylsulphamoyl)-1-methylethyl](phenyl)ketone

To a stirred solution of [1-(N,N-dimethylsulphamoyl)ethyl](phenyl)ketone (Reference Example 4; 33 mg, 0.14 mmol) in DMF was added potassium carbonate (39 mg, 0.28 mmol) and methyl iodide (60 mg, 0.42 mmol). The reaction was warmed to 400.0 and stirred at this temperature for 18 hours. Further methyl iodide was added (60 mg, 0.42 mmol) and the reaction was stirred at 40° C. for a further 24 hours. The volatiles were removed under reduced pressure and the resulting crude product was partitioned between ether and 1M sodium hydroxide solution, the ether layer was separated and re-extracted with sodium hydroxide solution then washed with brine, dried, filtered and evaporated to yield the product as a clear oil (16 mg, 43%). M/z): 256.

### Example 2

### (N,N-Dimethylsulphamoylmethyl)(pyrid-2-yl)ketone

To a solution of N-dimethylmethyl sulphonamide (1.23 g, 10 mmol) in THF (30 ml), under an inert atmosphere at 0° C. was added dropwise a solution of butyl lithium in hexanes 1.6M (12.5 ml, 20 mmol). After 30 minutes at 0° C., the mixture (white paste) was cooled to −78° C. and ethylpicolinate (1.51 g, 10 mmol) in THF (5 ml) was added. After 1 hour, the cooling bath was removed and the temperature was allowed to warn to 0° C. The mixture was diluted with cooled water and extracted twice with ether. The aqueous phase was acidified to pH 5 and extracted three times with EtOAc. The EtOAc extracts were combined and washed with brine, dried over, filtered and concentrated. The brown mauve resulting oil was triturated in ether until crystallisation occurred and the solid was filtered off (1.4 g, 66%). NMR: 2.92 (s, 6H), 4.98 (s, 2H), 7.55 (m, 1H), 7.89 (m, 1H), 8.11 (d, 1H), 8.74 (m, 1H); m/z: 229.

### Example 3

### N,N-Diisopropylsulphamoylmethyl)(4-fluorophenyl)ketone

To a stirred solution of N,N-diisopropylmethansulphonamide (120 mg, 0.67 mmol) in anhydrous THF (3 ml) at −20° C. was added a 1M solution of lithium bis(trimethylsilyl)amide in THF (1.34 ml, 1.34 mmol). The reaction was stirred at −20° C. for 30 minutes and then a solution of methyl-4-flurobenzoate (134 mg, 0.87 mmol) in anhydrous THF (1 ml) was added. The reaction was allowed to warm to room temperature over an hour then quenched with saturated ammonium chloride solution (5 ml). The layers were separated and the aqueous layer was extracted with EtOAc. The THF and EtOAc extracts were combined, washed with brine, dried, filtered and evaporated to yield an impure oil. The crude product was purified by column chromatography (eluting with DCM to 5% MeOH/DCM) to yield the product as an oil which crystallised on standing (70 mg, 35%). NMR: 1.25 (d, 12H), 3.65 (m, 2H), 4.40 (s, 2H), 7.10 (t, 2H), 8.02 (m, 2H); m/z: 300 (M−H)⁻.

### Examples 4-76 and Reference Examples 5 and 6

The procedure described in Example 3 was repeated using the appropriate reagent(s) in place of N,N-diisopropylmethanesulphonamide and/or methyl-4-flurobenzoate to give the following Examples. Where the methanesulphonamides were not known compounds or commercially available the preparation of the starting materials (SM) is indicated.

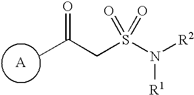

| Ex | Ring A | R¹ | R² | NMR | M/z | SM |
|---|---|---|---|---|---|---|
| 4 | 3-Chloro | Me | Me | 2.95 (s, 6H), 4.55 (s, 2H), | 262 | |
| | phenyl | | | 7.45 (t, 1H), 7.60 (m, 1H), | | |
| | | | | 7.90 (m, 1H), 8.00 (m, 1H) | | |
| 5 | 4-Fluoro | Et | Et | 1.20 (t, 6H), 3.30 (q, 4H), | 274 | ² |
| | phenyl | | | 4.50 (s, 2H), 7.15 (t, 2H), | | |
| | | | | 8.10 (m, 2H) | | |
| 6 | 3-Methoxy | Me | Me | 2.90 (s, 6H), 3.85 (s, 3H), | 258 | |
| | phenyl | | | 4.55 (s, 2H), 7.20 (m, 1H), | | |
| | | | | 7.40 (t, 1H), 7.50 (m, 1H), | | |
| | | | | 7.65 (m, 1H) | | |
| 7 | Fur-2-yl | i-Pr | i-Pr | 1.30 (d, 12H), 3.75 (m, | 272 | ³ |
| | | | | 2H), 4.40 (s, 2H), 6.60 (m, | (M − H⁻)⁻ | |
| | | | | 1H), 7.40 (m, 1H), 70 (m, | | |
| | | | | 1H) | | |
| 8 | 4-Fluoro | Me | —(CH₂)₂— | 2.95 (s, 3H), 3.25 (s, 3H), | 290 | Meth 1 |
| | phenyl | | OCH₃ | 3.35 (q, 2H), 3.44 (q, 2H), | | |
| | | | | 4.55 (s, 2H), 7.10 (t, 2H), | | |
| | | | | 8.00 (m, 2H) | | |
| 9 | 4-Fluoro | Me | Pr | 0.90 (t, 3H), 1.60 (m, 2H), | 274 | Meth 2 |
| | phenyl | | | 2.90 (s, 3H), 3.15 (t, 2H), | | |
| | | | | 4.55 (s, 2H), 7.15 (t, 2H), | | |
| | | | | 8.10 (m, 2H) | | |
| 10 | 4-Fluoro | Et | i-Pr | 1.20 (t, 3H), 1.25 (d, 6H), | 286 | Meth 3 |
| | phenyl | | | 3.20 (q, 2H), 3.95 (m, 1H), | (M − H⁻)⁻ | |
| | | | | 4.50 (s, 2H), 7.20 (t, 2H), | | |
| | | | | 8.10 (m, 2H) | | |
| 11 | 4-Methoxy | Me | Me | 2.90 (s, 6H), 3.90 (s, 3H), | 258 | |
| | phenyl | | | 3.55 (s, 2H), 7.00 (d, 2H), | | |
| | | | | 8.00 (d, 2H) | | |
| 12 | Thiazol-2-yl | Me | Me | 2.90 (s, 6H), 4.85 (s, 2H), | 233 | |
| | | | | 7.80 (d, 1H), 8.10 (d, 1H) | (M − H⁻)⁻ | |
| 13 | 1,2,3-Thia- | Me | Me | 2.95 (s, 6H), 4.50 (s, 2H), | 234 | |
| | diazol-5-yl | | | 9.20 (s, 1H) | (M − H⁻)⁻ | |
| 14 | Pyrazin-2-yl | Me | Me | 2.95 (s, 6H), 4.80 (s, 2H), | 228 | |
| | | | | 8.70 (m, 1H), 8.80 (d, 1H), | (M − H⁻)⁻ | |
| | | | | 9.30 (s, 1H) | | |
| 15 | 4-Fluoro | Me | —(CH₂)₂— | 2.30 (s, 6H), 2.50 (t, 2H), | 303 | Meth 6 |
| | phenyl | | N(Me)₂ | 3.00 (br s, 3H), 3.35 (t, | | |
| | | | | 2H), 4.70 (br s, 2H), 7.20 | | |
| | | | | (br t, 3H), 8.10 (m, 2H) | | |
| 16 | Thiazol-5-yl | Me | Me | 2.95 (s, 6H), 4.50 (s, 2H), | 235 | ¹ |
| | | | | 8.60 (s, 1H), 9.10 (s, 1H) | | |
| 17^{4,5} | 4-Trifluoro- | Me | Me | 2.9 (s, 6H), 4.6 (s, 2H), 7.8 | 294 | |
| | methyl | | | (d, 2H), 8.2 (d, 2H) | (M − H⁻)⁻ | |
| | phenyl | | | | | |
| 18^{4,5} | 4-t- | Me | Me | 1.6 (s, 9H), 2.9 (s, 6H), 4.6 | 282 | |
| | Butylphenyl | | | (s, 2H), 7.6 (d, 2H), 8.0 (d, | (M − H⁻)⁻ | |
| | | | | 2H) | | |
| 19⁴ | 6-Cyano | Me | Me | | 301 | |
| | naphth-2-yl | | | | (M − H⁻)⁻ | |
| 20^{4,5} | 4-Chloro-3- | Me | Me | 2.9 (s, 6H), 4.6 (s, 2H), 7.6 | 278 | |
| | fluorophenyl | | | (m, 1H), 7.8 (m, 2H) | (M − H⁻)⁻ | |
| 21⁴ | 3-Cyano-4- | Me | Me | 2.9 (s, 6H), 4.1 (s, 3H), 4.5 | | |
| | methoxy | | | (s, 2H), 7.1 (d, 1H), 8.3 (m, | | |
| | phenyl | | | 2H) | | |
| 22^{4,5} | 3-Iodo | Me | Me | 2.9 (s, 6H), 4.5 (s, 2H), 7.3 | 352 | |
| | phenyl | | | (dd, 1H), 8.0 (m, 2H), 8.4 | (M − H⁻)⁻ | |
| | | | | (d, 1H) | | |
| 23^{4,5} | 3-Iodo-4- | Me | Me | 2.9 (s, 6H), 4.0 (s, 3H), 4.5 | 382 | |
| | methoxy | | | (s, 2H), 6.9 (d, 1H), 8.0 | (M − H⁻)⁻ | |
| | phenyl | | | (dd, 1H), 8.4 (d, 2H) | | |
| 24^{4,5} | 3-Bromo | Me | Me | 2.9 (s, 6H), 4.5 (s, 2H), 7.4 | 306 | |
| | phenyl | | | (t, 1H), 7.7 (dd, 1H), 8.0 | (M − H⁻)⁻ | |
| | | | | (dd, 1H), 8.2 (d, 1H) | | |
| 25^{4,5} | Biphen-4-yl | Me | Me | | 302 | |
| | | | | | (M − H⁻)⁻ | |
| 26^{4,5} | 3,5- | Me | Me | | 254 | |
| | Dimethyl | | | | (M − H⁻)⁻ | |
| | phenyl | | | | | |
| 27^{4,5} | 3,5- | Me | Me | | 438 | |
| | Dibenzyloxy | | | | (M − H⁻)⁻ | |
| | phenyl | | | | | |
| RE | 4-Nitro | Me | Me | | 271 | |
| 5^{4,5} | phenyl | | | | (M − H⁻)⁻ | |
| 28^{4,5} | 3-Nitro | Me | Me | | 271 | |
| | phenyl | | | | (M − H⁻)⁻ | |
| 29^{4,5} | 3-Nitro-4- | Me | Me | | 305 | |
| | chlorophenyl | | | | (M − H⁻)⁻ | |
| 30^{4,5} | 6-Methyl | Me | Me | | 241 | |
| | pyrid-3-yl | | | | (M − H⁻)⁻ | |
| 31⁶ | 2,4-Difluoro | i-Pr | i-Pr | d⁶-DMSO, 1.22 (d, 12H), | 318 | ³ |
| | phenyl | | | 3.75 (m, 2H), 4.65 (s, 2H), | (M − H⁻)⁻ | |
| | | | | 7.24 (m, 1H), 7.42 (m, | | |
| | | | | 1H), 8.00 (m, 1H) | | |
| 32⁶ | 4-Bromo | Me | Me | d⁶-DMSO, 2.82 (s, 6H), | 304 | |
| | phenyl | | | 4.92 (s, 2H), 7.77 (d, 2H), | (M − H⁻)⁻ | |
| | | | | 7.96 (d, 2H) | | |
| 33⁶ | 4-Iodo | Me | Me | d⁶-DMSO, 2.80 (s, 6H), | 354 | |
| | phenyl | | | 4.88 (s, 2H), 7.78 (d, 2H), | | |
| | | | | 7.95 (d, 2H) | | |
| 34⁶ | 3-(Allyloxy) | Me | Me | d⁶-DMSO, 2.81 (s, 6H), | 284 | |
| | phenyl | | | 4.63 (m, 2H), 4.90 (s, 2H), | | |
| | | | | 5.27 (m, 1H), 5.41 (m, | | |
| | | | | 1H), 6.05 (m, 1H), 7.27 | | |
| | | | | (m, 1H), 7.46 (m, 1H), | | |
| | | | | 7.54 (m, 1H), 7.63 (m, 1H) | | |
| 35^{4,7} | 3-Cyano | i-Pr | i-Pr | | 307 | ³ |
| | phenyl | | | | (M − H⁻)⁻ | |
| 36^{4,7} | Phenyl | i-Pr | i-Pr | | 282 | ³ |
| | | | | | (M − H⁻)⁻ | |
| 37^{4,6} | 1,3-Benzo- | i-Pr | i-Pr | d⁶-DMSO, 1.22 (d, 12H), | 326 | ³ |
| | dioxol-5-yl | | | 3.74 (m, 2H), 4.66 (s, 2H), | (M − H⁻)⁻ | |
| | | | | 6.15 (s, 2H), 7.04 (d, 1H), | | |
| | | | | 7.46 (d, 1H), 7.66 (dd, 1H) | | |
| 38^{4,7} | 4-Cyano | i-Pr | i-Pr | | 307 | |
| | phenyl | | | | (M − H⁻)⁻ | |
| 39^{4,6} | 4- | i-Pr | i-Pr | | 353 | ³ |
| | (Acetamido- | | | | (M − H⁻)⁻ | |
| | methyl) | | | | | |
| | phenyl | | | | | |
| 40^{4,7} | 1,1,3- | i-Pr | i-Pr | | 387 | ^{3,9} |
| | Trioxo-2,3- | | | | (M − H⁻)⁻ | |
| | dihydro-1,2- | | | | | |
| | benziso- | | | | | |
| | thiazol-6-yl | | | | | |
| 41^{4,7} | 1H-indol-5- | i-Pr | i-Pr | d⁶-DMSO, 1.23 (d, 12H), | 321 | ³ |
| | yl | | | 3.76 (m, 2H), 4.72 (s, 2H), | (M − H⁻)⁻ | |
| | | | | 6.63 (m, 1H), 7.14 (br s, | | |
| | | | | 1H), 7.47 (m, 2H), 7.76 | | |
| | | | | (m, 1H), 8.38 (m, 1H) | | |
| 42^{4,7} | 4- | Me | Me | d⁶-DMSO, 2.79 (s, 6H), | 334 | |
| | (Benzyloxy) | | | 4.81 (s, 2H), 5.23 (s, 2H), | | |
| | phenyl | | | 7.15 (d, 2H), 7.41 (m, 5H), | | |
| | | | | 8.00 (d, 2H) | | |
| 43^{4,6} | Biphen-3-yl | Me | Me | | 304 | |
| 44^{4,6} | 3-Acetyl | Me | Me | d⁶-DMSO, 2.64 (s, 3H), | 270 | |
| | phenyl | | | 2.83 (s, 6H), 5.00 (s, 2H), | | |
| | | | | 7.71 (m, 1H), 8.24 (m, | | |
| | | | | 2H), 8.50 (m, 1H) | | |
| 45^{4,6} | 3- | Me | Me | | 332 | |
| | (Benzyloxy) | | | | (M − H⁻)⁻ | |
| | phenyl | | | | | |
| 46^{4,6} | 4,5-Dichloro | Me | Me | d⁶-DMSO, 2.83 (s, 6H), | 303 | |
| | thiazol-2-yl | | | 4.88 (s, 2H) | (M − H⁻)⁻ | |
| 47^{4,6} | Benzothien- | Me | Me | d⁶-DMSO, 2.84 (s, 6H), | 282 | |
| | 2-yl | | | 4.95 (s, 2H), 7.53 (m, 2H), | (M − H⁻)⁻ | |
| | | | | 8.06 (m, 2H), 8.57 (s, 1H) | | |
| 48^{4,6} | 2-Chloro | Me | Me | | 266 | |
| | thien-5-yl | | | | (M − H⁻)⁻ | |
| 49^{4,6} | 3-Chloro-4- | Me | Me | d⁶-DMSO, 2.21 (s, 3H), | 280 | |
| | methylthien- | | | 2.84 (s, 6H), 4.79 (s, 2H), | (M − H⁻)⁻ | |
| | 2-yl | | | 7.92 (s, 1H) | | |
| 50^{4,6} | 5-(4-Chloro | Me | Me | | 326 | ¹⁰ |
| | phenyl) | | | | (M − H⁻)⁻ | |
| | pyrazol-3-yl | | | | | |
| 51^{4,5} | 4-(3- | Me | Me | | 296 | |
| | Oxobutyl) | | | | | |
| | phenyl | | | | | |
| 52^{4,5} | 4-(2-Chloro | Me | Me | d⁶-DMSO, 2.81 (s, 6H), | 355 | |
| | pyrimidin-4- | | | 4.82 (s, 2H), 6.90 (d, 1H), | | |
| | ylamino) | | | 781 (d, 2H), 8.05 (d, 2H), | | |
| | phenyl | | | 8.27 (d, 2H), 10.48 (br s, | | |
| | | | | 1H) | | |
| 53^{4,5} | 4- | Me | Me | d⁶-DMSO, 2.81 (s, 6H), | 305 | |
| | Sulphamoyl | | | 4.98 (s, 2H), 7.57 (s, 2H), | (M − H⁻)⁻ | |
| | phenyl | | | 7.96 (d, 2H), 8.22 (d, 2H) | | |
| 54^{4,8} | Benzothien- | i-Pr | —(CH₂)₂—O- | | 382 | Meth |
| | 2-yl | | iPr | | (M − H⁻)⁻ | 11 |
| 55^{4,8} | 4-Fluoro | i-Pr | —(CH₂)₂—O- | | 344 | Meth |
| | phenyl | | iPr | | (M − H⁻)⁻ | 11 |
| 56^{4,8} | 2,4-Difluoro | i-Pr | —(CH₂)₂—O- | | 362 | Meth |
| | phenyl | | iPr | | (M − H⁻)⁻ | 11 |
| 57^{4,8} | 2-Chloro | i-Pr | —(CH₂)₂—O- | | 366 | Meth |
| | thien-5-yl | | iPr | | (M − H⁻)⁻ | 11 |
| 58^{4,8} | Thiazol-2-yl | i-Pr | —(CH₂)₂—O- | | 333 | Meth |
| | | | iPr | | (M − H⁻)⁻ | 11 |
| 59^{4,8} | 4-Fluoro | i-Pr | —(CH₂)₂OCH₃ | | 316 | Meth |
| | phenyl | | | | (M − H⁻)⁻ | 10 |
| 60^{4,8} | 2-Chloro | i-Pr | —(CH₂)₂OCH₃ | | 338 | Meth |
| | thien-5-yl | | | | (M − H⁻)⁻ | 10 |
| 61^{4,8} | 2,4-Difluoro | i-Pr | —(CH₂)₂OCH₃ | | 334 | Meth |
| | phenyl | | | | (M − H⁻)⁻ | 10 |
| 62 | 4-(t-Butyl | i-Pr | i-Pr | 0.27 (s, 6H), 1.00 (s, 9H), | | ¹¹ |
| | dimethyl | | | 1.33 (d, 12H), 3.73 (m, | | |
| | silyloxy) | | | 2H), 4.47 (s, 2H), 6.90 (d, | | |
| | phenyl | | | 2H), 7.99 (d, 2H) | | |
| RE | Cyclohexyl | Me | Me | 1.3 (m, 5H), 1.7 (d, 1H), | | |
| 6^{4,5} | | | | 1.8 (b, 2H), 1.9 (b, 2H), 2.7 | | |
| | | | | (m, 1H), 2.9 (s, 6H), 4.0 (s, | | |
| | | | | 2H) | | |
| 63⁴ | Cyclohex-1- | Me | Me | | 232 | |
| | en-1-yl | | | | (M + H)⁺ | |
| 64⁷ | 3-Phenyl | Me | Me | d⁶-DMSO, 1.80 (m, 6H), | | |
| | cyclopentyl | | | 2.27 (m, 1H), 2.80 (s, 6H), | | |
| | | | | 3.04 (m, 1H), 4.37 (s, 2H), | | |
| | | | | 7.22 (m, 5H) | | |
| 65⁷ | 1,2,3,4- | Me | Me | d⁶-DMSO, 1.62 (m, 1H), | | |
| | Tetrahydro | | | 2.14 (m, 1H), 2.80 (s, 6H), | | |
| | naphth-2-yl | | | 2.93 (m, 5H), 4.50 (m, | | |
| | | | | 2H), 7.07 (m, 4H) | | |
| 66⁷ | Cyclopentyl | Me | Me | d⁶-DMSO, 1.54 (m, 4H), | | |
| | | | | 1.66 (m, 2H), 1.80 (m, | | |
| | | | | 2H), 2.77 (s, 6H), 3.10 (m, | | |
| | | | | 1H), 4.34 (s, 2H) | | |
| 67⁷ | 1-(Pyrid-4- | Me | Me | d⁶-DMSO, 1.43 (m, 2H), | | |
| | yl)- | | | 1.92 (m, 2H), 2.78 (s, 6H), | | |
| | piperidin-4- | | | 2.90 (m, 3H), 3.92 (m, | | |
| | yl | | | 2H), 4.42 (s, 2H), 6.80 (m, | | |
| | | | | 2H), 8.12 (m, 2H) | | |
| 68⁷ | 4-Tetrahyro | Me | Me | d⁶-DMSO, 1.43 (m, 2H), | | |
| | pyran-4-yl | | | 1.77 (m, 2H), 2.77 (s, 6H), | | |
| | | | | 2.86 (m, 1H), 3.33 (m, | | |
| | | | | 2H), 3.85 (m, 2H), 4.39 (s, | | |
| | | | | 2H) | | |
| 69⁷ | 1-Oxo | Me | Me | d⁶-DMSO, 1.23 (m, 2H), | | |
| | octahydro | | | 2.05 (m, 2H), 2.16 (m, | | |
| | pyrido[1,2-a] | | | 1H), 2.37 (m, 2H), 2.76 (s, | | |
| | pyrazin-7-yl | | | 6H), 2.86 (m, 2H), 3.05 | | |
| | | | | (m, 2H), 3.22 (m, 1H), | | |
| | | | | 4.40 (m, 2H), 7.60 (br s, | | |
| | | | | 1H) | | |
| 70 | Benzothien- | i-Pr | i-Pr | 1.30-1.23 (d, 6H); 3.68-3.84 | +ve 340 | ³ |
| | 2-yl | | | (m, 2H); 4.56 (s, 2H); | | |
| | | | | 7.37-7.53 (m, 2H); | | |
| | | | | 7.90 (d, 1H); 7.91-7.99 (d, | | |
| | | | | 1H); 8.22 (s, 1H) | | |
| 71 | Benzthiazol- | i-Pr | i-Pr | 1.26-1.45 (d, 12H); 3.74-3.94 | +ve 341 | ³ |
| | 2-yl | | | 3.94 (m, 2H); 4.92 (s, 2H) | | |
| | | | | 7.50 (m, 2H); 7.93-8.02 (d, | | |
| | | | | 1H); 8.17-8.27 (d, 1H) | | |
| 72 | 3- | i-Pr | i-Pr | 1.28-1.45 (m, 18H); 3.67-3.83 | +ve 342 | ³ |
| | Isopropoxy | | | 3.83 (m, 2H); 4.51 (s, 2H); | | |
| | phenyl | | | 4.55-4.69 (m, 1H); 7.08-7.16 | | |
| | | | | (app dd, 1H); 7.33-7.43 | | |
| | | | | (app t, 1H), 7.55 (m, 1H); | | |
| | | | | 7.59-7.65 (d, 1H) | | |
| 73 | Thiazol-2-yl | i-Pr | i-Pr | 1.23-1.43 (d, 12H); 3.72-3.91 | +ve 291 | ³ |
| | | | | 3(m, 2H); 4.80 (s, 2H); | | |
| | | | | 7.71-7.79 (d, 1H); | | |
| | | | | (8.03-8.10 (d, 1H) | | |
| 74 | 4-Bromo | i-Pr | i-Pr | 1.09-1.32 (d, 12H); 3.66-3.86 | −ve 360 | ³ |
| | phenyl | | | 3.86 (m, 2H); 4.76 (s, 2H); | | |
| | | | | 7.71-7.85 (d, 2H); | | |
| | | | | 7.90-8.03 (d, 2H) | | |
| 75 | 4- | i-Pr | i-Pr | 1.23-1.40 (d, 12H); 1.40-1.42 | +ve 342 | ³ |
| | Isopropoxy | | | 1.42 (d, 6H); 3.66-3.80 (m, | | |
| | phenyl | | | 2H); 4.48 (s, 2H); 4.60-4.72 | | |
| | | | | (m, 1H); 6.89-6.96 (d, | | |
| | | | | 2H); 7.97-8.07 (d, 2H) | | |
| 76⁶ | 4-Methyl | Me | Me | d⁶-DMSO, 2.40 (s, 3H), | 242 | |
| | Phenyl | | | 2.80 (s, 6H), 4.84 (s, 2H), | (M + H)⁺ | |
| | | | | 7.36 (d, 2H), 7.92 (d, 2H) | | |

¹Starting ester prepared according to Tetrahedron Lett.; EN; 25; 51; 1984; 5939-5942
²Sulphonamide preparation: J. Amer. Chem. Soc.; 76; 1954; 303
³Sulphonamide preparation: Tetrahedron; EN; 25; 1969; 181-189
⁴Reaction carried out at room temperature overnight
⁵Product crystallised from EtOAc/hexane in place of chromatography.
⁶Purification by chromatography (eluting with 25% EtOAc/isohexane to 50% EtOAc/isohexane)
⁷Product triturated with diethyl ether.
⁸Purification by chromatography (eluting with 10% EtOAc/isohexane to 20% EtOAc/isohexane)
⁹Starting ester prepared according to Azerbaidzhanshii Khimicheskii Zhurnal; 1997; 1-4; 62-66.
¹⁰Starting ester prepared according to PCT Int. Appl.; 1997; 99pp.; WO9721682.
¹¹Starting ester prepared according to J. Org. Chem.; 1991; 56(16); 4884-7.

⁶Purification by chromatography (eluting with 25% EtOAc/isohexane to 50% EtOAc/isohexane) ⁷Product triturated with diethyl ether. ⁸Purification by chromatography (eluting with 10% EtOAc/isohexane to 20% EtOAc/isohexane) ⁹Starting ester prepared according to Azerbaidzhanskii Khimicheskii Zhumal; 1997; 1-4; 62-66. ¹⁰ Starting ester prepared according to PCT Int. Appl.; 1997; 99 pp.; WO9721682. ¹¹ Starting ester prepared according to J. Org. Chem.; 1991; 56(16); 4884-7.

### Examples 77-79

The procedure described in Example 3 was repeated using the appropriate reagent(s) in place of N,N-diisopropylmethanesulphonamide to give the following Examples.

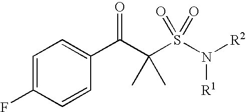

| Ex | R¹ | R² | NMR | M/z | SM |
|---|---|---|---|---|---|
| 77 | Me | Me | 1.73 (s, 6H), 2.93 (s, 6H), 7.10 (m, 2H), 8.05 (m, 2H) | | Method 7 |
| 78 | Me | i-Pr | 1.12 (d, 6H), 1.62 (s, 6H), 2.68 (s, 3H), 3.87 (m, 1H), | 302 | Method 8 |
| | | | 7.30 (m, 2H), 7.94 (m, 2H) | | |
| 79 | H | i-Pr | 1.28 (d, 6H), 2.10 (s, 6H), 3.80 (m, 1H), 7.07 (m, | | Method 9 |
| | | | 2H), 7.87 (m, 2H), 11.40 (br s, 1H) | | |

### Example 80

### (N,N-Dimethylsulphamoylmethyl)(4-chlorophenyl)ketone

To a stirred solution of methyl-4-chlorobenzoate (500 mg, 2.94 mmol) and N,N-dimethylmethanesulphonamide (302 mg, 2.45 mmol) in ethylene glycol dimethyl ether (15 ml) was added NaH (60% suspension in mineral oil, 265 mg, 6.62 mmol). The reaction was warmed to 85° C. and stirred at this temperature for 3 hours. The reaction was cooled to room temperature and then quenched with water (˜40 ml). The water was extracted with ether then the ether was extracted with 1M NaOH. The aqueous fractions were combined and acidified to ˜pH3 by the addition of concentrated HCl. The resulting suspension was extracted with DCM (2×40 m]), the DCM layers were combined, washed with water and brine then dried, filtered and evaporated to yield an oil. This oil was purified by column chromatography (20 g Si, DCM to 1% MeOH/DCM) to yield a solid (325 mg, 42%). NMR (DMSO-d₆): 2.95 (s, 6H), 4.55 (s, 2M), 7.50 (d, 2H), 8.00 (d, 2H); m/z 262 [M+H]⁺.

### Example 81

### [2-(N,N-Diisopropylsulphamoyl)-2-(methyl)ethyl](4-fluorophenyl)ketone

Methyl iodide (51 μl, 0.825 mmol) was added to a stirred mixture of (N,N-diisopropylsulphamoylmethyl)(4-fluorophenyl)ketone (Example 3; 100 mg, 0.33 mmol) and potassium carbonate (114 mg, 0.825 mmol) in DMF (5 ml) at room temperature under an inert atmosphere. The reaction mixture was stirred overnight before quenching with water (50 ml) and then extraction with EtOAc (2×50 ml). The organics were washed with brine (50 ml) and then dried over magnesium sulphate. The solvent was then removed under reduced pressure and the resulting brown gum was purified by chromatography (eluting with 10% EtOAc/isohexane) to yield a colourless oil which solidified on scratching (57 mgs, 53%). NMR (DMSO-d₆): 1.22 (d, 12H), 1.62 (s, 6H), 3.61 (m, 2H), 7.28 (m, 2H), 8.02 (m, 2H).

### Examples 82-90

The procedure described in Example 81 was repeated using the appropriate starting material(s) in place of (N,N-diisopropylsulphamoylmethyl)(4-fluorophenyl)ketone (Example 3) to give the following Examples.

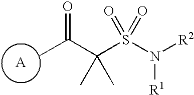

| Ex | Ring A | R¹ | R² | NMR | M/z | SM |
|---|---|---|---|---|---|---|
| 82 | 2,4-Difluorophenyl | i-Pr | i-Pr | 1.36 (d, 12H), 1.65 | | Example 31 |
| | | | | (s, 6H), 3.72 (m, | | |
| | | | | 2H), 6.87 (m, 2H), | | |
| | | | | 7.90 (m, 1H) | | |
| 83 | 3-Chlorophenyl | Me | Me | | 290 | Example 4 |
| 84 | 4-Chloro-3-fluorophenyl | Me | Me | | 308 | Example 20 |
| 85 | 4-Trifluoro-methylphenyl | Me | Me | 1.64 (s, 6H), 2.91 (s, | 324 | Example 17 |
| | | | | 6H), 7.83 (m, 2H), | | |
| | | | | 7.93 (m, 211) | | |
| 86 | 4-Cyanophenyl | i-Pr | i-Pr | 1.33 (d, 12H), 1.71 | | Example 38 |
| | | | | (s, 6H), 3.64 (m, | | |
| | | | | 2H), 7.72 (m, 2H), | | |
| | | | | 8.12 (m, 2H) | | |
| 87 | 4-Fluorophenyl | Me | Pr | | 302 | Example 9 |
| 88 | 4-Fluorophenyl | Me | —(CH₂)₂— | | 318 | Example 8 |
| | | | OMe | | | |
| 89 | 2,4-Difluorophenyl | i-Pr | —(CH₂)₂— | 1.34 (d, 6H), 1.65 (s, | 364 | Example 61 |
| | | | OMe | 6H), 3.35 (s, 3H), | | |
| | | | | 3.40 (m, 2H), 3.53 | | |
| | | | | (m, 2H), 3.97 (m, | | |
| | | | | 1H), 6.87 (m, 2H), | | |
| | | | | 7.77 (m, 1H) | | |
| 90 | 4-Fluorophenyl | i-Pr | —(CH₂)₂— | | 374 | Example 55 |
| | | | O-iPr | | | |

### Examples 91-93

The procedure described in Example 81 was repeated using the appropriate starting material(s) in place of (N,N-diisopropylsulphamoylmethyl)(4-fluorophenyl)ketone (Example 3) to give the following Examples.

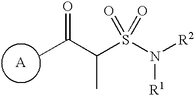

| Ex | Ring A | R¹ | R² | M/z | SM |
|---|---|---|---|---|---|
| 91 | 4-Fluorophenyl | i-Pr | —(CH₂)₂—O-iPr | 358 (M − H⁻)⁻ | Example 55 |
| 92 | 2-Chlorothien-5-yl | i-Pr | —(CH₂)₂—OMe | 352 (M − H⁻)⁻ | Example 60 |
| 93 | Benzothien-2-yl | i-Pr | —(CH₂)₂—O-iPr | 396 (M − H⁻)⁻ | Example 54 |

### Example 94

### [2-(N,N-Diisopropylsulphamoyl)ethyl][4-(t-butyldimethylsilyloxy phenyl]ketone

Methyl iodide (2.33 ml, 37.5 mmol) was added to a stirred mixture of (N,N-diisopropylsulphamoylmethyl)[4-(t-butyldimethylsilyloxy)phenyl]ketone (Example 62; 7.5 mmol) and potassium carbonate (5.18 g, 37.5 mmol) in acetone (60 ml) at room temperature under an inert atmosphere. The reaction mixture was stirred overnight at room temperature before adding more methyl iodide (2.33 ml, 37.5 mmol) and heating at reflux for 1 hour. The reaction mixture was quenched with water (200 ml) and then extracted with EtOAc (2×150 ml). The organics were dried over magnesium sulphate before being removed under reduced pressure. The resulting orange oil was purified by chromatography (eluting with 10% EtOAc/isohexane) to yield a colourless oil (1.542 g, 48%). NMR: 0.15 (s, 6H), 0.89 (s, 12H), 1.18 (m, 12H), 1.55 (d, 3H), 3.58 (m, 2H), 4.84 (q, 1H), 6.79 (d, 2H), 7.87 (d, 2H).

### Example 95

### (N,N-Dimethylsulphamoylmethyl)(3-methylphenyl)ketone

N,N-dimethylaminomethanesulphonamide (37 mg, 0.3 mmol) and anhydrous THF (3 ml) were placed in a tube. To this solution was added a 1M solution of lithium bis(trimethylsilyl)amide in THF (0.6 ml, 0.6 mmol). The reaction was allowed to stir at room temperature for 30 minutes. At this point a solution of ethyl 3-methylbenzoate (60 mg, 0.36 mmol) in anhydrous THF (1 ml) was added. The reaction was stirred at room temperature for 2 hours then quenched with sat ammonium chloride solution (2 ml). The tube was capped then shaken and allowed to settle. The organic layer was collected and evaporated under reduced pressure, the resulting crude material was purified by prep LCMS (140% over 9.5 mins, acetonitrile/water, with a constant 5 ml/min 4% formic acid/acetonitrile) to yield a solid (29 mg, 40%). M/z: 242.

### Examples 96-106

The procedure described in Example 95 was repeated using the appropriate ester in place of ethyl 3-methylbenzoate.

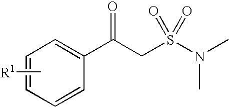

| Ex | R¹ | NMR | MS |
|---|---|---|---|
| 96 | 3-CH₂═CHCH₂— | | 268 |
| 97 | 4-CN | | 253 |
| 98 | 3-F | 3.00 (s, 6H), 4.50 (s, 2H), 7.35 (m, 1H), 7.50 | 246 |
| | | (m, 1H), 7.75 (m, 1H), 7.85 (d, 1H) | |
| 99 | 3-CN | | 253 |
| 100 | 4-MeC(O)— | | 268 |
| | | | (M − H⁻)⁻ |
| 101 | 2-F | | 246 |
| 102 | 2,4-diF | 2.95 (s, 6H), 4.60 (s, 2H), 6.90 (m, 1H), 7.00 | 262 |
| | | (m, 1H), 7.95 (m, 1H) | (M − H⁻)⁻ |
| 103 | 3-MeO, 4-Cl | | 290 |
| | | | (M − H⁻)⁻ |
| 104 | 4-HC≡C— | | 252 |
| 105 | 4-CH₂═CH—CH₂—O— | | 284 |
| 106 | 3,4,5-triMeO- | NMR (DMSO-d₆): 2.80 (s, 6H), 3.80 (s, | 318 |
| | | 3H), 3.85 (s, 6H), 4.95 (s, 2H), 7.35 (s, 2H) | |

### Example 107

### (4-Fluorophenyl)-[1-(N,N-diiospropelsulphamoyl)cyclopentyl]ketone

1,4-Dibromobutane (373 mgs, 1.7 mmol) was added drop wise to a stirred mixture of (N,N-diisopropylsulphamoylmethyl)(4-fluorophenyl)ketone (Example 3; 400 mgs, 1.33 mmol) and potassium carbonate (460 mgs, 3.3 mmol) in dimethylformamide (5 ml). After stirring overnight the reaction mixture was quenched with water and extracted with EtOAc. The EtOAc phase was washed with water, brine and dried (MgSO₄). Removal of the EtOAc gave a brown oil which was chromatographed (9 g silica cartridge, eluent 10% EtOAc in Hexane) to give the required product (80 mgs). NMR (DMSO-d₆) 1.15-1.37 (d, 12H), 1.37-1.59 (m, 2H), 1.65-1.92 (m, 2H), 2.49-2.74 (m, 4H), 3.50-3.65 (m, 21), 7.02-7.19 (m, 2H), 8.17-8.32 (m, 2H). m/z: 340.14 (M-CH₃).

Preparation of Starting Materials

The starting materials for the Examples above are either commercially available or are readily prepared by standard methods from known materials. For example the following reactions are illustrations but not limitations of the preparation of some of the starting materials used in the above reactions.

Method 1

### 1-Methoxy-2-[N-(methylmesylamino]ethyl

To a stirred solution of N-(2-methoxyethyl)methylamine (750 mg, 8.43 mmol) and triethylamine (938 mg, 9.27 mmol) in anhydrous DCM (60 ml) at 0° C. was added mesylchloride (966 mg, 8.43 mmol). The reaction was stirred at 0° C. for 10 minutes then allowed to warm to room temperature and left to stir for a further 30 minutes. The reaction was then transferred to a separating funnel and washed with 2M HCl (20 ml), water (20 ml) and brine (20 ml) then dried, filtered and evaporated to yield the product as a pale yellow oil (935 mg, 67%). NMR: 2.85 (s, 3H), 2.95 (s, 3H), 3.40 (m, 5H), 3.55 (t, 2H).

Methods 2-3

The procedure described in Method 1 was repeated using the appropriate amine in place of N-(2-methoxyethyl)methylamine.

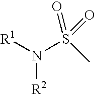

| Meth | R¹ | R² | NMR |
|---|---|---|---|
| 2 | Me | Pr | 0.95 (t, 3H), 1.60 (m, 2H), 2.80 (s, 3H), 2.95 (s, 3H), 3.10 (t, |
| | | | 2H) |
| 3 | Et | i-Pr | 1.25 (m, 9H), 2.85 (s, 3H), 3.20 (q, 2H), 4.10 (m, 1H) |
| 4 | H | —(CH₂)₂—OCH₃ | 2.99 (s, 3H), 3.31 (m, 2H), 3.40 (s, 3H), 3.53 (m, 2H), 4.92 |
| | | | (br s, 1H) |
| 5 | H | —(CH₂)₂—O-iPr | 1.17 (d, 6H), 2.99 (s, 3H), 3.28 (m, 2H), 3.55 (t, 2H), 3.62 |
| | | | (m, 1H), 4.82 (br s, 1H) |

Method 6

### 1-(N,N-Dimethylamino)-2-[N-(methyl)mesylamino]ethyl

To a stirred solution of N,N,N-trimethylethylenediamine (1.02 g, 10 mmol) and triethylamine (1.11 g, 11 mmol) in anhydrous DCM (70 ml) at 0° C. was added mesylchloride (1.15 g, 10 mmol). The reaction was stirred at 0° C. for 10 minutes then allowed to warm to room temperature and left to stir for a further 30 minutes. Volatiles removed under reduced pressure and resulting oil taken up in DCM (60 ml) then washed with 2M NaOH (30 ml) and brine (30 ml). The solvent was removed under reduced pressure to yield the product as an oil (962 mg, 53%). NMR: 2.30 (s, 6H), 2.50 (t, 2H), 2.85 (s, 3H), 2.90 (s, 3H), 3.30 (t, 2H).

Method 7

### Propane-2-sulphonic acid dimethyl amide

To a stirred solution of 2M dimethylamine in THF (5.35 ml, 10.7 mmol) and pyridine (865 μl, 10.7 mmol) in anhydrous THF (10 ml) was added isopropylsulphonylchloride (1 ml, 8.9 mmol). The reaction was stirred at room temperature overnight. Volatiles removed under reduced pressure and resulting oil taken up in EtOAc (60 ml) then washed with 0.5M HCl (30 ml) and water (30 ml). The solvent was dried over magnesium sulphate and then removed under reduced pressure to yield the product as an orange oil (688 mg, 51%). NMR: 1.36 (d, 6H), 2.93 (s, 6H), 3.25 (m, 1H).

Methods 8-9

The procedure described in Method 7 was repeated using the appropriate amine in place of dimethylamine.

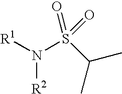

| Meth | R¹ | R² | NMR |
|---|---|---|---|
| 8 | Me | i-Pr | (DMSO-d₆) 1.10 (d, 6H), 1.18 (d, 6H), 2.68 (s, 3H), 3.24 (m, 1H), 3.94 |
| | | | (m, 1H) |
| 9 | H | i-Pr | (DMSO-d₆) 1.05 (d, 6H), 1.10 (d, 6H), 3.06 (m, 1H), 3.40 (m, 1H), |
| | | | 6.80 (d, 1H) |

Method 10

### N-Isopropyl-N-(2-methoxy-ethyl)-methanesulphonamide

To a stirred solution of N-(2-methoxy-ethyl)-methanesulphonamide (Method 4) (1.2 g, 7.7 mmol) in DMF (20 ml) under an inert atmosphere was added sodium hydride (400 mg, 10 mmol). 2-Bromopropane (1.73 ml, 18.48 mmol) was added and the reaction heated at 70° C. for 7 h. The reaction mixture was quenched with water (100 ml) and then extracted into EtOAc (100 ml). The organics were further washed with brine (100 ml) before being dried over magnesium sulphate. The solvent was then removed under reduced pressure to yield the product as a yellow oil (600 mg, 40%). NMR: 1.26 (d, 6H), 2.88 (s, 3H), 3.31 (m, 2H), 3.38 (s, 3H), 3.55 (m, 2H), 4.09 (m, 1H).

Method 11

### N-Isopropyl-N-(2-isopropoxy-ethyl)-methanesulphonamide

The procedure described in Method 10 was repeated using N-(2-isopropoxyethyl)methanesulphonamide (Method 5) in place of N-(2-methoxy-ethyl)-methanesulphonamide. NMR: 1.16 (d, 6H), 1.27 (d, 6H), 2.90 (s, 3H), 3.29 (m, 2H), 3.56 (m, 3H), 4.09 (m, 1H).

1. A method for inhibiting 11βHSD1, comprising administering a compound of formula (I): 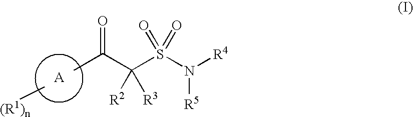
wherein
Ring A is selected from carbocyclyl or heterocyclyl;
each R¹ is independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino, tri-(C₁₋₄alkyl)silyloxy, carbocyclyl, heterocyclyl, carbocyclylC₀₋₄alkylene-Y—, and heterocyclylC₀₋₄alkylene-Y—; wherein R¹ may be optionally substituted on carbon with one or more R⁶ groups; and wherein if said heterocyclyl contains an —NH— moiety, that nitrogen may be optionally substituted with an R⁷ group;
n is 0-5;
R² and R³ are independently selected from hydrogen, hydroxy, amino, cyano, C₁₋₄alkyl, C₁₋₄alkoxy, N-(C₁₋₄alkyl)amino, N,N-(C₁₋₄alkyl)₂amino, carbocyclyl, heterocyclyl, carbocyclylC₁₋₄alkyl, and heterocyclylC₁₋₄alkyl; or R² and R³ together are C₂₋₆alkylene; wherein R² and R³ may be independently optionally substituted on carbon with one or more R⁸ groups; and wherein if said heterocyclyl contains an —NH— moiety, that nitrogen may be optionally substituted with an R⁹ group;
one of R⁴ and R⁵ is C₁₋₄ alkyl and the other is selected from hydrogen and C₁₋₄alkyl; wherein R⁴ and R⁵ may be optionally substituted on carbon with one or more R¹⁰ groups;
Y is selected from —S(O)ₐ—, —O—, —NR¹²—, —C(O), —C(O)NR¹³—, —NR¹⁴C(O)—, and —SO₂NR¹⁵—; wherein a is 0 to 2;
R¹², R¹³, R¹⁴ and R¹⁵ are independently selected from hydrogen, phenyl and C₁₋₄alkyl;
R⁶ and R⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino, carbocyclyl, and heterocyclyl; wherein R⁶ and R⁸ may be independently optionally substituted on carbon with one or more R¹¹ groups;
R¹⁰ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, and C₁₋₄alkylsulphonylamino; wherein R¹⁰ may be independently optionally substituted on carbon with one or more R¹⁶ groups;
R⁷ and R⁹ are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)₂carbamoyl, benzyl, benzyloxycarbonyl, benzoyl, and phenylsulphonyl;
R¹¹ and R¹⁶ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, and N-methyl-N-ethylsulphamoyl; or a pharmaceutically acceptable salt thereof.

2. A method according to claim 1 wherein Ring A is selected from pyridyl, phenyl, thienyl, furyl, pyrazinyl, 1,2,3-thiadiazolyl, thiazolyl, cyclohexyl, naphthyl, cyclohexenyl, pyrazolyl, benzothienyl, indolyl, 1,1,3-trioxo-2,3-dihydro-1,2-benzisothiazolyl, 1,3-benzodioxolyl, cyclopentyl, tetrahydropyranyl, 1-oxooctahydropyrido[1,2-a]pyrazinyl, 1,2,3,4-tetrahydronaphthyl, piperidinyl, and benzthiazolyl.

3. A method according to either of claims 1 wherein each R¹ is independently selected from halo, nitro, cyano, sulphamoyl, C₁₋₄alkyl, C₂₋₄alkenyl, C₂₋₄alkynyl, C₁₋₄alkoxy, C₁₋₄alkanoyl, tri-(C₁₋₄alkyl)silyloxy, carbocyclyl, and heterocyclylC₀₋₄alkylene-Y—; wherein R¹ may be optionally substituted on carbon with one or more R⁶ groups;
Y is —NR¹²—;
R¹² is hydrogen; and
R⁶ is selected from halo, C₂₋₄alkenyl, C₁₋₄alkanoyl, C₁₋₄alkanoylamino, and carbocyclyl.

4. A method according to claim 1, wherein n is 0-2.

5. A method according to claim 1, wherein R² and R³ are independently selected from hydrogen and C₁₋₄alkyl; or R² and R³ together are C₂₋₆alkylene.

6. A method according to claim 1, wherein
R¹⁰ is selected from C₁₋₄alkoxy and N,N-(C₄alkyl)₂amino.

7. A method of claim 1,
wherein
Ring A is selected from carbocyclyl and heterocyclyl;
each R¹ is independently selected from halo, nitro, cyano, sulphamoyl, C₁₋₄alkyl, C₂₋₄alkenyl, C₂₋₄alkynyl, C₁₋₄alkoxy, C₁₋₄alkanoyl, tri-(C₁₋₄alkyl)silyloxy, carbocyclyl, and heterocyclylC₀₋₄alkylene-Y—; wherein R¹ may be optionally substituted on carbon with one or more R⁶ groups;
Y is —NR¹²—;
R¹² is hydrogen;
R⁶ is selected from halo, C₂₋₄alkenyl, C₁₋₄alkanoyl, C₁₋₄alkanoylamino, and carbocyclyl;
n is 0-3;
R² and R³ are independently selected from hydrogen and C₁₋₄alkyl, or R² and R³ together are C₂₋₆alkylene;
one of R⁴ and R⁵ is selected from hydrogen and C₁₋₄alkyl and the other is C₁₋₄alkyl; wherein R⁴ and R⁵ may be optionally substituted on carbon with one or more R¹⁰ groups; and
R¹⁰ is selected from C₁₋₄alkoxy and N,N-(C₁₋₄alkyl)₂amino;
or a pharmaceutically acceptable salt thereof.

8. A compound selected from:
(4-fluorophenyl)[N-(2-methoxyethyl)-N-(methyl)sulphamoylmethyl]ketone;
(2,4-difluorophenyl)[1-(N,N-diisopropylsulphamoyl)-1 methylethyl]ketone;
(2,4-difluorophenyl)(N,N-diisopropylsulphamoylmethyl)ketone;
(thiazol-2-yl)(N,N-dimethysulphamoylmethyl)ketone;
(4-fluorophenyl)[N-(2-isopropoxyethyl)-N-(isopropyl)sulphamoylmethyl]ketone;
(pyrazin-2-yl)(N,N-dimethysulphamoylmethyl)ketone;
(4-isopropoxyphenyl)(N,N-diisopropylsulphamoylmethyl)ketone;
(3-cyanophenyl)(N,N-diisopropylsulphamoylmethyl)ketone; and
(pyrid-2-yl)(N,N-dimethysulphamoylmethyl)ketone;
or a pharmaceutically acceptable salt thereof.

9. A compound of formula (Ia): 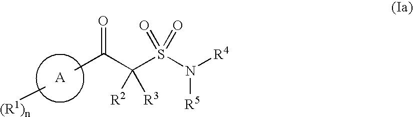
wherein
Ring A is selected from phenyl, pyridyl, thiazolyl, thienyl, and furyl;
each R¹ is independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, and C₁₋₄alkylsulphonylamino; wherein R¹ may be optionally substituted on carbon with one or more R⁶ groups; and wherein if said heterocyclyl contains an —NH— moiety, that nitrogen may be optionally substituted with an R⁷ group;
n is 0-3;
R² and R³ are independently selected from hydrogen, hydroxy, amino, cyano, C₁₋₄alkyl, C₁₋₄alkoxy, N-(C₁₋₄alkyl)amino, N,N-(C₁₋₄alkyl)₂amino, carbocyclyl, heterocyclyl, carbocyclylC₁₋₄alkyl, and heterocyclylC₁₋₄alkyl; wherein R² and R³ may be independently optionally substituted on carbon with one or more R⁸ groups; and wherein if said heterocyclyl contains an —NH— moiety, that nitrogen may be optionally substituted with an R⁹ group selected from;
R⁴ and R⁵ are independently C₁₋₄alkyl; wherein R⁴ and R⁵ may be optionally substituted on carbon with one or more R¹⁰ groups;
R⁶ and R⁸ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, and C₁₋₄alkylsulphonylamino; wherein R⁶ and R⁸ may be independently optionally substituted on carbon with one or more R¹¹ groups;
R¹⁰ is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, and C₁₋₄alkylsulphonylamino; wherein R¹⁰ may be independently optionally substituted on carbon with one or more R¹⁶ groups;
R⁷ and R⁹ are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)₂carbamoyl, benzyl, benzyloxycarbonyl, benzoyl, and phenylsulphonyl;
R¹¹ and R¹⁶ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, and N-methyl-N-ethylsulphamoyl;
or a pharmaceutically acceptable salt thereof;
with the proviso that said compound is not (N-methyl-N-butylsulphamoylmethyl)(phenyl)ketone; [1-(N,N-dimethylsulphamoyl)ethyl](phenyl)ketone; (N,N-dimethylsulphamoylmethyl)(4-nitrophenyl)ketone; (N,N-dimethylsulphamoylmethyl)(4-fluoro-2-methylaminophenyl)ketone; (N,N-dimethylsulphamoylmethyl)(3-methoxy-4-methyl-6-aminophenyl)ketone; (N,N-dimethylsulphamoylmethyl)(3-methoxy-6-aminophenyl)ketone; (N,N-dimethylsulphamoylmethyl)(phenyl)ketone; (N,N-dimethylsulphamoylmethyl)(2-nitro-4-methoxyphenyl)ketone; (N,N-dimethylsulphamoylmethyl)(2-amino-4-methoxyphenyl)ketone; [1-(N-methyl-N-butylsulphamoyl)ethyl](phenyl)ketone; or (N,N-dimethylsulphamoylmethyl)(thien-2-yl)ketone.

10. A pharmaceutical composition which comprises a compound of claim 8 or 9 in association with a pharmaceutically acceptable diluent or carrier.

11-13. (canceled)

14. A method for the treatment of a metabolic syndrome, comprising inhibiting 11βHSD1 according to claim 1.

15. A method for the treatment of diabetes, obesity, hyperlipidaemia, hyperglycaemia hyperinsulinemia or hypertension, comprising inhibiting 11βHSD1 according to claim 1.

16. A method for the treatment of glaucoma, osteoporosis, tuberculosis, dementia, cognitive disorders or depression, comprising inhibiting 11βHSD1 according to claim 1.

17. (canceled)
